SEQUENCE LISTING (12 sequences)

SEQ ID NO: 1 - DNA, 1584 bp, synthetic construct
  source 1..1584: mol_type = other DNA
atgaccaccgcaccggttgccgataaaattgccgaaaccccgaccgttaccgcagcaaataatgtgcgcccgatgaccggcgcagaatatctggaaagtctgcgcgatggtcgtgaaatttatattcgtggtgaacgtgtggaagatgttaccgaacatccggcctttcgtaatagtgcacgtagtgtggcccgtatgtatgatgcactgcatgaaccggcagcacagggtgtgctgagcgttccgaccgataccggtaatggcggttttacccatccgttttttaaaaccgcacataatagcgatgatctgatggccgcacgtgatgcaattgttgcctggcagcgtgaagtttatggttggctgggccgtagcccggattataaagcaagttttctgggcaccctgggcgcaaatagcgatttttatggcgaatataaacagaatgcactggattggtataaaaaaagtcaggaaagtgttctgtacctgaatcatgccattgtgaatccgccgattgatcgtgccaaaccggcagatgaaaccgcagatgtgtgcgttcatgttgtggaagaaaccgatgcaggcttaattgtgagtggtgcaaaagtggttgccaccggtagtgcaattaccaatgcaaattttattgcccattacggtctgccgctgcgcaaaaaagaatttggtctgatttttaccgtgccgatggatagtccgggcctgaaactgctgtgccgcaccagttatgaaatgaatgccgcagcaaccggtacaccgtttgattatccgctgagcagtcgttttgatgaaaatgatagcattatggttttcgatcgcgttctggtgccgtgggaaaatgtttttgcatacgatgcagaaaccaccaataattttgtgatgcgcagcggctttctgaatcgctttatgtttcatggctgcgcccgcctggcagttaaactggattttattgccggctgtgtgatgaaaggcgtggaaatgaccggtacagccggttttcgcggcgtgcagatgcagattggcgaaattctgaattggcgcgatatgttttggggtctgagtgatgcaatggctaaaagcccggatgaatgggtgaatggcgccgttcagccgaatctgaattatggtctggcatatcgcacctttatgggtgtgggctatccgcgcattaaagaaattattcagcaggtgctgggcagcggcctgatttatctgaatagtcatgcagatgattggaaaaatcctgatattgaaccgtatctgaatcagtatgtgcgtggtagcgatggtattgccgcaattgatcgcgttcagctgctgaaactgttatgggatgccgttggtacagaatttggtggccgccatgaactgtatgaacgtaattatggtggtgatcatgaagccgttcgctttcagaccctgtttgcatatcaggccagcggtcaggatgccgccctgaaaggctttgcagaacagtgtatgagcgaatatgatgttgatggttggacccgtcctgatctgattaataatgatgatctggaaatcgtgtggaatcgtaaataa

SEQ ID NO: 2 - protein, 527 aa, synthetic construct
MTTAPVADKIAETPTVTAANNVRPMTGAEYLESLRDGREIYIRGERVEDVTEHPAFRNSARSVARMYDALHEPAAQGVLSVPTDTGNGGFTHPFFKTAHNSDDLMAARDAIVAWQREVYGWLGRSPDYKASFLGTLGANSDFYGEYKQNALDWYKKSQESVLYLNHAIVNPPIDRAKPADETADVCVHVVEETDAGLIVSGAKVVATGSAITNANFIAHYGLPLRKKEFGLIFTVPMDSPGLKLLCRTSYEMNAAATGTPFDYPLSSRFDENDSIMVFDRVLVPWENVFAYDAETTNNFVMRSGFLNRFMFHGCARLAVKLDFIAGCVMKGVEMTGTAGFRGVQMQIGEILNWRDMFWGLSDAMAKSPDEWVNGAVQPNLNYGLAYRTFMGVGYPRIKEIIQQVLGSGLIYLNSHADDWKNPDIEPYLNQYVRGSDGIAAIDRVQLLKLLWDAVGTEFGGRHELYERNYGGDHEAVRFQTLFAYQASGQDAALKGFAEQCMSEYDVDGWTRPDLINNDDLEIVWNRK

SEQ ID NO: 3 - DNA, 1809 bp, synthetic construct
  source 1..1809: mol_type = other DNA
atggccccagttaaacaagactttaacatagacgttcaaacaatcgagaacactgacatctctttgtctgaatacatatatcttaggatagcccaattgggtgtcaagtcgatctttggtgtcccaggtgacttcaacctgaatcttgtggatgaactagacaaagttccccaattgaaatggataggatgttgtaatgagctaaatgccacttatgctgctgacggctatgcaaaagcatcaggaacgataggcgttgtggtcactacttatggtgtgggtgagctaagcgccatcaacggtatagcaggagcattcgccgaatatgctcctgttcttcacattgtaggcacctccgctatggcaacaaaacgacttgaacatgtgcacaacattcatcatcttgcagggtctaagaatttcttggatagaccagaccattatatatacgaaaaaatggttgatgacatctgcattgtcaaagagtctttaagcgagatcgaaaatgcctgtggccagatagataatgccatcgtacagacttatctgctttccagaccaggatacctatttttacctagaaatatggccactatgaaagttccaagagaaaggctattcaaccaaccattagccttggaaagagttgatcttcaccccggtgaaacgctacaagttgttgagaagatcttggaaaagttctatcatgcaaaagaaccggccttgattgtggattatttaaccagaccctttagaatgatggaaaactgctccaagttgatcggtgctttggagaataaagtcaacattttcagcagaccaatgtccaaaggctttgtcgatgagagccacccaagatatattggctgttatattggaaaacaatccaagcacccaagtactagtgatattctagaaaaaaagagcgatttcattctgagtgtgggaacgtttgatgttgaaacgaataacggaggcttcacatccaagcttccccaagaacatttggtggaattgaaccctcattttactcgtgttggaactcaatgcttttcaaatgttaacatgtgccatgtcctcccactcctggcaagtaagctgagaggtgatttgatcagcatggccacagttcatccaaatgatttcagcctcagaaagaaagaaaaggcacaggataaaatgaaggctctcaaccagagccatcttgtaaaatctacagagctactcctaaatgcaaacgacactttgatagttgaaacttgctcatttatgtttgcagttccagatatcgcgttccctaacaatacacaatttatcagtcagtcattctacaactccataggatacgcacttcccgccaccttgggtgtcagcattgcaaagcgtgatttcagaaaaccaggaaaagtcgtacttattcaaggggatggctctgctcaaatgaccattcaagaactggctaccatggtcagacaaaaggtcaaacccaccattttactcctcaacaacgaaggatacacagttgaacgaatgattctaggtccaaccaaagaatataacgatatagctcctaattgggattggactggaatgctgagagcattcggcgatatacgtggccattccaagagcatctcaattgacacatgtggtagattagataagctcgttcaaactcgagagtttcaagaacctacacacctaaattttgtggaacttatcttgggcagaatggacgccccggaaaggtttgccaatatggtaaaagaaattgctaacttagagcacgcaagtaaaagtattcattag

SEQ ID NO: 4 - protein, 602 aa, synthetic construct
MAPVKQDFNIDVQTIENTDISLSEYIYLRIAQLGVKSIFGVPGDFNLNLVDELDKVPQLKWIGCCNELNATYAADGYAKASGTIGVVVTTYGVGELSAINGIAGAFAEYAPVLHIVGTSAMATKRLEHVHNIHHLAGSKNFLDRPDHYIYEKMVDDICIVKESLSEIENACGQIDNAIVQTYLLSRPGYLFLPRNMATMKVPRERLFNQPLALERVDLHPGETLQVVEKILEKFYHAKEPALIVDYLTRPFRMMENCSKLIGALENKVNIFSRPMSKGFVDESHPRYIGCYIGKQSKHPSTSDILEKKSDFILSVGTFDVETNNGGFTSKLPQEHLVELNPHFTRVGTQCFSNVNMCHVLPLLASKLRGDLISMATVHPNDFSLRKKEKAQDKMKALNQSHLVKSTELLLNANDTLIVETCSFMFAVPDIAFPNNTQFISQSFYNSIGYALPATLGVSIAKRDFRKPGKVVLIQGDGSAQMTIQELATMVRQKVKPTILLLNNEGYTVERMILGPTKEYNDIAPNWDWTGMLRAFGDIRGHSKSISIDTCGRLDKLVQTREFQEPTHLNFVELILGRMDAPERFANMVKEIANLEHASKSIH

SEQ ID NO: 5 - DNA, 510 bp, synthetic construct
  source 1..510: mol_type = other DNA
atgatgaccgtttatgatagcgcactgacaatggaagaaaccaccctgcgtgatgcaatgagccgttttgcaaccggtgttagcgttgttaccgttggtggtgaacatacacatggtatgaccgcaaatgcctttacctgtgttagcctggatccgcctctggttctgtgttgtgttgcacgtaaagcaaccatgcatgcagcaattgaaggtgcacgtcgttttgcagttagcgttatgggtggtgatcaagaacgtaccgcacgttattttgcagataaacgtcgtccgcgtggtcgtgcacagtttgatgttgttgattggcagcctggtccgcatacaggtgcaccgctgctgagcggtgcgctggcatggctggaatgtgaagttgcacagtggcatgaaggtggcgatcataccatttttctgggtcgtgttctgggttgtcgtcgtggtccggatagtccggcactgctgttttatggtagcgattttcatcagatccgctaa

SEQ ID NO: 6 - protein, 169 aa, synthetic construct
MMTVYDSALTMEETTLRDAMSRFATGVSVVTVGGEHTHGMTANAFTCVSLDPPLVLCCVARKATMHAAIEGARRFAVSVMGGDQERTARYFADKRRPRGRAQFDVVDWQPGPHTGAPLLSGALAWLECEVAQWHEGGDHTIFLGRVLGCRRGPDSPALLFYGSDFHQIR

SEQ ID NO: 7 - DNA, 1053 bp, synthetic construct
  source 1..1053: mol_type = other DNA
atgaattatcagaacgacgatttacgcatcaaagaaatcaaagagttacttcctcctgtcgcattgctggaaaaattccccgctactgaaaatgccgcgaatacggttgcccatgcccgaaaagcgatccataagatcctgaaaggtaatgatgatcgcctgttggttgtgattggcccatgctcaattcatgatcctgtcgcggcaaaagagtatgccactcgcttgctggcgctgcgtgaagagctgaaagatgagctggaaatcgtaatgcgcgtctattttgaaaagccgcgtaccacggtgggctggaaagggctgattaacgatccgcatatggataatagcttccagatcaacgacggtctgcgtatagcccgtaaattgctgcttgatattaacgacagcggtctgccagcggcaggtgagtttctcaatatgatcaccccacaatatctcgctgacctgatgagctggggcgcaattggcgcacgtaccaccgaatcgcaggtgcaccgcgaactggcatcagggctttcttgtccggtcggcttcaaaaatggcaccgacggtacgattaaagtggctatcgatgccattaatgccgccggtgcgccgcactgcttcctgtccgtaacgaaatgggggcattcggcgattgtgaataccagcggtaacggcgattgccatatcattctgcgcggcggtaaagagcctaactacagcgcgaagcacgttgctgaagtgaaagaagggctgaacaaagcaggcctgccagcacaggtgatgatcgatttcagccatgctaactcgtccaaacaattcaaaaagcagatggatgtttgtgctgacgtttgccagcagattgccggtggcgaaaaggccattattggcgtgatggtggaaagccatctggtggaaggcaatcagagcctcgagagcggggagccgctggcctacggtaagagcatcaccgatgcctgcatcggctgggaagataccgatgctctgttacgtcaactggcgaatgcagtaaaagcgcgtcgcgggtaa

SEQ ID NO: 8 - protein, 350 aa, synthetic construct
MNYQNDDLRIKEIKELLPPVALLEKFPATENAANTVAHARKAIHKILKGNDDRLLVVIGPCSIHDPVAAKEYATRLLALREELKDELEIVMRVYFEKPRTTVGWKGLINDPHMDNSFQINDGLRIARKLLLDINDSGLPAAGEFLNMITPQYLADLMSWGAIGARTTESQVHRELASGLSCPVGFKNGTDGTIKVAIDAINAAGAPHCFLSVTKWGHSAIVNTSGNGDCHIILRGGKEPNYSAKHVAEVKEGLNKAGLPAQVMIDFSHANSSKQFKKQMDVCADVCQQIAGGEKAIIGVMVESHLVEGNQSLESGEPLAYGKSITDACIGWEDTDALLRQLANAVKARRG

SEQ ID NO: 9 - DNA, 1122 bp, synthetic construct
  source 1..1122: mol_type = other DNA
atggttgctgaattgaccgcattacgcgatcaaattgatgaagtcgataaagcgctgctgaatttattagcgaagcgtctggaactggttgctgaagtgggcgaggtgaaaagccgctttggactgcctatttatgttccggagcgcgaggcatctattttggcctcgcgtcgtgcagaggcggaagctctgggtgtaccgccagatctgattgaggatgttttgcgtcgggtgatgcgtgaatcttactccagtgaaaacgacaaaggatttaaaacactttgtccgtcactgcgtccggtggttatcgtcggcggtggcggtcagatgggacgcctgttcgagaagatgctgaccctctcgggttatcaggtgcggattctggagcaacatgactgggatcgagcggctgatattgttgccgatgccggaatggtgattgttagtgtgccaatccacgttactgagcaagttattggcaaattaccgcctttaccgaaagattgtattctggtcgatctggcatcagtgaaaaatgggccattacaggccatgctggtggcgcatgatggtccggtgctggggctacacccgatgttcggtccggacagcggtagcctggcaaagcaagttgtggtctggtgtgatggacgtaaaccggaagcataccaatggtttctggagcaaattcaggtctggggcgctcggctgcatcgtattagcgccgtcgagcacgatcagaatatggcgtttattcaggcactgcgccactttgctacttttgcttacgggctgcacctggcagaagaaaatgttcagcttgagcaacttctggcgctctcttcgccgatttaccgccttgagctggcgatggtcgggcgactgtttgctcaggatccgcagctttatgccgacatcattatgtcgtcagagcgtaatctggcgttaatcaaacgttactataagcgtttcggcgaggcgattgagttgctggagcagggcgataagcaggcgtttattgacagtttccgcaaggtggagcactggttcggcgattacgtacagcgttttcagagtgaaagccgcgtgttattgcgtcaggcgaatgacaatcgccagtaa

SEQ ID NO: 10 - protein, 373 aa, synthetic construct
MVAELTALRDQIDEVDKALLNLLAKRLELVAEVGEVKSRFGLPIYVPEREASILASRRAEAEALGVPPDLIEDVLRRVMRESYSSENDKGFKTLCPSLRPVVIVGGGGQMGRLFEKMLTLSGYQVRILEQHDWDRAADIVADAGMVIVSVPIHVTEQVIGKLPPLPKDCILVDLASVKNGPLQAMLVAHDGPVLGLHPMFGPDSGSLAKQVVVWCDGRKPEAYQWFLEQIQVWGARLHRISAVEHDQNMAFIQALRHFATFAYGLHLAEENVQLEQLLALSSPIYRLELAMVGRLFAQDPQLYADIIMSSERNLALIKRYYKRFGEAIELLEQGDKQAFIDSFRKVEHWFGDYVQRFQSESRVLLRQANDNRQ

SEQ ID NO: 11 - DNA, 4454 bp, synthetic construct
  source 1..4454: mol_type = other DNA
ggcagtgagcgcaacgcaattaatgtgagttagctcactcattaggcaccatcgtttaggcaccccaggctttacactttatgcttccggctcgtataatgtgtggaattgtgagcggataacaatttcaactataagaaggagatatacatatgaattatcagaacgacgatttacgcatcaaagaaatcaaagagttacttcctcctgtcgcattgctggaaaaattccccgctactgaaaatgccgcgaatacggttgcccatgcccgaaaagcgatccataagatcctgaaaggtaatgatgatcgcctgttggttgtgattggcccatgctcaattcatgatcctgtcgcggcaaaagagtatgccactcgcttgctggcgctgcgtgaagagctgaaagatgagctggaaatcgtaatgcgcgtctattttgaaaagccgcgtaccacggtgggctggaaagggctgattaacgatccgcatatggataatagcttccagatcaacgacggtctgcgtatagcccgtaaattgctgcttgatattaacgacagcggtctgccagcggcaggtgagtttctcaatatgatcaccccacaatatctcgctgacctgatgagctggggcgcaattggcgcacgtaccaccgaatcgcaggtgcaccgcgaactggcatcagggctttcttgtccggtcggcttcaaaaatggcaccgacggtacgattaaagtggctatcgatgccattaatgccgccggtgcgccgcactgcttcctgtccgtaacgaaatgggggcattcggcgattgtgaataccagcggtaacggcgattgccatatcattctgcgcggcggtaaagagcctaactacagcgcgaagcacgttgctgaagtgaaagaagggctgaacaaagcaggcctgccagcacaggtgatgatcgatttcagccatgctaactcgtccaaacaattcaaaaagcagatggatgtttgtgctgacgtttgccagcagattgccggtggcgaaaaggccattattggcgtgatggtggaaagccatctggtggaaggcaatcagagcctcgagagcggggagccgctggcctacggtaagagcatcaccgatgcctgcatcggctgggaagataccgatgctctgttacgtcaactggcgaatgcagtaaaagcgcgtcgcgggtaacgcagcaggaggttaagatggttgctgaattgaccgcattacgcgatcaaattgatgaagtcgataaagcgctgctgaatttattagcgaagcgtctggaactggttgctgaagtgggcgaggtgaaaagccgctttggactgcctatttatgttccggagcgcgaggcatctattttggcctcgcgtcgtgcagaggcggaagctctgggtgtaccgccagatctgattgaggatgttttgcgtcgggtgatgcgtgaatcttactccagtgaaaacgacaaaggatttaaaacactttgtccgtcactgcgtccggtggttatcgtcggcggtggcggtcagatgggacgcctgttcgagaagatgctgaccctctcgggttatcaggtgcggattctggagcaacatgactgggatcgagcggctgatattgttgccgatgccggaatggtgattgttagtgtgccaatccacgttactgagcaagttattggcaaattaccgcctttaccgaaagattgtattctggtcgatctggcatcagtgaaaaatgggccattacaggccatgctggtggcgcatgatggtccggtgctggggctacacccgatgttcggtccggacagcggtagcctggcaaagcaagttgtggtctggtgtgatggacgtaaaccggaagcataccaatggtttctggagcaaattcaggtctggggcgctcggctgcatcgtattagcgccgtcgagcacgatcagaatatggcgtttattcaggcactgcgccactttgctacttttgcttacgggctgcacctggcagaagaaaatgttcagcttgagcaacttctggcgctctcttcgccgatttaccgccttgagctggcgatggtcgggcgactgtttgctcaggatccgcagctttatgccgacatcattatgtcgtcagagcgtaatctggcgttaatcaaacgttactataagcgtttcggcgaggcgattgagttgctggagcagggcgataagcaggcgtttattgacagtttccgcaaggtggagcactggttcggcgattacgtacagcgttttcagagtgaaagccgcgtgttattgcgtcaggcgaatgacaatcgccagtaagcggccgccaccgctgagcaataactagcataaccccttggggcctctaaacgggtcttgaggggttttttgctgaaaggaggaactatatccgggtaacgaattcaagcttgatatcattcaggacgagcctcagactccagcgtaactggactgcaatcaactcactggctcaccttcacgggtgggcctttcttcggtagaaaatcaaaggatcttcttgagatcctttttttctgcgcgtaatctgctgcttgcaaacaaaaaaaccaccgctaccagcggtggtttgtttgccggatcaagagctaccaactctttttccgaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcatcgatttttgtgatgctcgtcaggggggcggagcctatggaaaaacgccagcaacgcagaaaggcccacccgaaggtgagccaggtgattacatttgggccctcatcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcatttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatagctcctgaaaatctcgataactcaaaaaatacgcccggtagtgatcttatttcattatggtgaaagttggaacctcttacgtgccgatcaagtcaaaagcctccggtcggaggcttttgactttctgctatggaggtcaggtatgatttaaatggtcagtattgagcgatatctagagaattcgtc

SEQ ID NO: 12 - DNA, 8548 bp, synthetic construct
  source 1..8548: mol_type = other DNA
ggcagtgagcgcaacgcaattaatgtgagttagctcactcattaggcaccatcgtttaggcaccccaggctttacactttatgcttccggctcgtataatgtgtggaattgtgagcggataacaatttcaactataagaaggagatatacatatgaattatcagaacgacgatttacgcatcaaagaaatcaaagagttacttcctcctgtcgcattgctggaaaaattccccgctactgaaaatgccgcgaatacggttgcccatgcccgaaaagcgatccataagatcctgaaaggtaatgatgatcgcctgttggttgtgattggcccatgctcaattcatgatcctgtcgcggcaaaagagtatgccactcgcttgctggcgctgcgtgaagagctgaaagatgagctggaaatcgtaatgcgcgtctattttgaaaagccgcgtaccacggtgggctggaaagggctgattaacgatccgcatatggataatagcttccagatcaacgacggtctgcgtatagcccgtaaattgctgcttgatattaacgacagcggtctgccagcggcaggtgagtttctcaatatgatcaccccacaatatctcgctgacctgatgagctggggcgcaattggcgcacgtaccaccgaatcgcaggtgcaccgcgaactggcatcagggctttcttgtccggtcggcttcaaaaatggcaccgacggtacgattaaagtggctatcgatgccattaatgccgccggtgcgccgcactgcttcctgtccgtaacgaaatgggggcattcggcgattgtgaataccagcggtaacggcgattgccatatcattctgcgcggcggtaaagagcctaactacagcgcgaagcacgttgctgaagtgaaagaagggctgaacaaagcaggcctgccagcacaggtgatgatcgatttcagccatgctaactcgtccaaacaattcaaaaagcagatggatgtttgtgctgacgtttgccagcagattgccggtggcgaaaaggccattattggcgtgatggtggaaagccatctggtggaaggcaatcagagcctcgagagcggggagccgctggcctacggtaagagcatcaccgatgcctgcatcggctgggaagataccgatgctctgttacgtcaactggcgaatgcagtaaaagcgcgtcgcgggtaacgcagcaggaggttaagatggttgctgaattgaccgcattacgcgatcaaattgatgaagtcgataaagcgctgctgaatttattagcgaagcgtctggaactggttgctgaagtgggcgaggtgaaaagccgctttggactgcctatttatgttccggagcgcgaggcatctattttggcctcgcgtcgtgcagaggcggaagctctgggtgtaccgccagatctgattgaggatgttttgcgtcgggtgatgcgtgaatcttactccagtgaaaacgacaaaggatttaaaacactttgtccgtcactgcgtccggtggttatcgtcggcggtggcggtcagatgggacgcctgttcgagaagatgctgaccctctcgggttatcaggtgcggattctggagcaacatgactgggatcgagcggctgatattgttgccgatgccggaatggtgattgttagtgtgccaatccacgttactgagcaagttattggcaaattaccgcctttaccgaaagattgtattctggtcgatctggcatcagtgaaaaatgggccattacaggccatgctggtggcgcatgatggtccggtgctggggctacacccgatgttcggtccggacagcggtagcctggcaaagcaagttgtggtctggtgtgatggacgtaaaccggaagcataccaatggtttctggagcaaattcaggtctggggcgctcggctgcatcgtattagcgccgtcgagcacgatcagaatatggcgtttattcaggcactgcgccactttgctacttttgcttacgggctgcacctggcagaagaaaatgttcagcttgagcaacttctggcgctctcttcgccgatttaccgccttgagctggcgatggtcgggcgactgtttgctcaggatccgcagctttatgccgacatcattatgtcgtcagagcgtaatctggcgttaatcaaacgttactataagcgtttcggcgaggcgattgagttgctggagcagggcgataagcaggcgtttattgacagtttccgcaaggtggagcactggttcggcgattacgtacagcgttttcagagtgaaagccgcgtgttattgcgtcaggcgaatgacaatcgccagtaagcggccgccaccgctgagcaataactagcataaccccttggggcctctaaacgggtcttgaggggttttttgctgaaaggaggaactatatccatcgtttaggcaccccaggctttacactttatgcttccggctcgtataatgtgtggaattgtgagcggataacaatttcaactataagaaggagatatacatatggccccagttaaacaagactttaacatagacgttcaaacaatcgagaacactgacatctctttgtctgaatacatatatcttaggatagcccaattgggtgtcaagtcgatctttggtgtcccaggtgacttcaacctgaatcttgtggatgaactagacaaagttccccaattgaaatggataggatgttgtaatgagctaaatgccacttatgctgctgacggctatgcaaaagcatcaggaacgataggcgttgtggtcactacttatggtgtgggtgagctaagcgccatcaacggtatagcaggagcattcgccgaatatgctcctgttcttcacattgtaggcacctccgctatggcaacaaaacgacttgaacatgtgcacaacattcatcatcttgcagggtctaagaatttcttggatagaccagaccattatatatacgaaaaaatggttgatgacatctgcattgtcaaagagtctttaagcgagatcgaaaatgcctgtggccagatagataatgccatcgtacagacttatctgctttccagaccaggatacctatttttacctagaaatatggccactatgaaagttccaagagaaaggctattcaaccaaccattagccttggaaagagttgatcttcaccccggtgaaacgctacaagttgttgagaagatcttggaaaagttctatcatgcaaaagaaccggccttgattgtggattatttaaccagaccctttagaatgatggaaaactgctccaagttgatcggtgctttggagaataaagtcaacattttcagcagaccaatgtccaaaggctttgtcgatgagagccacccaagatatattggctgttatattggaaaacaatccaagcacccaagtactagtgatattctagaaaaaaagagcgatttcattctgagtgtgggaacgtttgatgttgaaacgaataacggaggcttcacatccaagcttccccaagaacatttggtggaattgaaccctcattttactcgtgttggaactcaatgcttttcaaatgttaacatgtgccatgtcctcccactcctggcaagtaagctgagaggtgatttgatcagcatggccacagttcatccaaatgatttcagcctcagaaagaaagaaaaggcacaggataaaatgaaggctctcaaccagagccatcttgtaaaatctacagagctactcctaaatgcaaacgacactttgatagttgaaacttgctcatttatgtttgcagttccagatatcgcgttccctaacaatacacaatttatcagtcagtcattctacaactccataggatacgcacttcccgccaccttgggtgtcagcattgcaaagcgtgatttcagaaaaccaggaaaagtcgtacttattcaaggggatggctctgctcaaatgaccattcaagaactggctaccatggtcagacaaaaggtcaaacccaccattttactcctcaacaacgaaggatacacagttgaacgaatgattctaggtccaaccaaagaatataacgatatagctcctaattgggattggactggaatgctgagagcattcggcgatatacgtggccattccaagagcatctcaattgacacatgtggtagattagataagctcgttcaaactcgagagtttcaagaacctacacacctaaattttgtggaacttatcttgggcagaatggacgccccggaaaggtttgccaatatggtaaaagaaattgctaacttagagcacgcaagtaaaagtattcattagcgcagcaggaggttaagatgaccaccgcaccggttgccgataaaattgccgaaaccccgaccgttaccgcagcaaataatgtgcgcccgatgaccggcgcagaatatctggaaagtctgcgcgatggtcgtgaaatttatattcgtggtgaacgtgtggaagatgttaccgaacatccggcctttcgtaatagtgcacgtagtgtggcccgtatgtatgatgcactgcatgaaccggcagcacagggtgtgctgagcgttccgaccgataccggtaatggcggttttacccatccgttttttaaaaccgcacataatagcgatgatctgatggccgcacgtgatgcaattgttgcctggcagcgtgaagtttatggttggctgggccgtagcccggattataaagcaagttttctgggcaccctgggcgcaaatagcgatttttatggcgaatataaacagaatgcactggattggtataaaaaaagtcaggaaagtgttctgtacctgaatcatgccattgtgaatccgccgattgatcgtgccaaaccggcagatgaaaccgcagatgtgtgcgttcatgttgtggaagaaaccgatgcaggcttaattgtgagtggtgcaaaagtggttgccaccggtagtgcaattaccaatgcaaattttattgcccattacggtctgccgctgcgcaaaaaagaatttggtctgatttttaccgtgccgatggatagtccgggcctgaaactgctgtgccgcaccagttatgaaatgaatgccgcagcaaccggtacaccgtttgattatccgctgagcagtcgttttgatgaaaatgatagcattatggttttcgatcgcgttctggtgccgtgggaaaatgtttttgcatacgatgcagaaaccaccaataattttgtgatgcgcagcggctttctgaatcgctttatgtttcatggctgcgcccgcctggcagttaaactggattttattgccggctgtgtgatgaaaggcgtggaaatgaccggtacagccggttttcgcggcgtgcagatgcagattggcgaaattctgaattggcgcgatatgttttggggtctgagtgatgcaatggctaaaagcccggatgaatgggtgaatggcgccgttcagccgaatctgaattatggtctggcatatcgcacctttatgggtgtgggctatccgcgcattaaagaaattattcagcaggtgctgggcagcggcctgatttatctgaatagtcatgcagatgattggaaaaatcctgatattgaaccgtatctgaatcagtatgtgcgtggtagcgatggtattgccgcaattgatcgcgttcagctgctgaaactgttatgggatgccgttggtacagaatttggtggccgccatgaactgtatgaacgtaattatggtggtgatcatgaagccgttcgctttcagaccctgtttgcatatcaggccagcggtcaggatgccgccctgaaaggctttgcagaacagtgtatgagcgaatatgatgttgatggttggacccgtcctgatctgattaataatgatgatctggaaatcgtgtggaatcgtaaataacgcagcaggaggttaagatgatgaccgtttatgatagcgcactgacaatggaagaaaccaccctgcgtgatgcaatgagccgttttgcaaccggtgttagcgttgttaccgttggtggtgaacatacacatggtatgaccgcaaatgcctttacctgtgttagcctggatccgcctctggttctgtgttgtgttgcacgtaaagcaaccatgcatgcagcaattgaaggtgcacgtcgttttgcagttagcgttatgggtggtgatcaagaacgtaccgcacgttattttgcagataaacgtcgtccgcgtggtcgtgcacagtttgatgttgttgattggcagcctggtccgcatacaggtgcaccgctgctgagcggtgcgctggcatggctggaatgtgaagttgcacagtggcatgaaggtggcgatcataccatttttctgggtcgtgttctgggttgtcgtcgtggtccggatagtccggcactgctgttttatggtagcgattttcatcagatccgctaagcggccgccaccgctgagcaataactagcataaccccttggggcctctaaacggggggtaacgaattcaagcttgatatcattcaggacgagcctcagactccagcgtaactggactgcaatcaactcactggctcaccttcacgggtgggcctttcttcggtagaaaatcaaaggatcttcttgagatcctttttttctgcgcgtaatctgctgcttgcaaacaaaaaaaccaccgctaccagcggtggtttgtttgccggatcaagagctaccaactctttttccgaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcatcgatttttgtgatgctcgtcaggggggcggagcctatggaaaaacgccagcaacgcagaaaggcccacccgaaggtgagccaggtgattacatttgggccctcatcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcatttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatagctcctgaaaatctcgataactcaaaaaatacgcccggtagtgatcttatttcattatggtgaaagttggaacctcttacgtgccgatcaagtcaaaagcctccggtcggaggcttttgactttctgctatggaggtcaggtatgatttaaatggtcagtattgagcgatatctagagaattcgtc

(19) United States
(12) Patent Application Publication
(10) Pub. No.: US 2025/0207157 A1
(43) Pub. Date: Jun. 26, 2025

(54) METHODS OF PRODUCING HYDROXYTYROSOL

(71) Applicant: Conagen Inc., Bedford, MA (US)

(72) Inventors: Rui Zhou, Acton, MA (US); Oliver Yu, Lexington, MA (US); Steven Chen, Coto de Caza, CA (US)

(73) Assignee: Conagen Inc., Bedford, MA (US)

(21) Appl. No.: 18/791,545

(22) Filed: Aug. 1, 2024

Related U.S. Application Data
(63) Continuation of application No. PCT/US2023/061733, filed on Feb. 1, 2023.
(60) Provisional application No. 63/305,657, filed on Feb. 1, 2022.

(51) Int. Cl. C12P 7/22; C12N 9/10; C12N 9/90; C12N 15/70

(52) U.S. Cl. CPC C12P 7/22; C12N 9/1085; C12N 9/90; C12N 15/70; C12Y 205/01054; C12Y 504/99005; C12N 2800/101

(57) ABSTRACT

Provided herein is a method of producing hydroxytyrosol. The method includes: culturing a recombinant host microorganism in a medium, wherein: the host microorganism has been transformed with: a first polynucleotide sequence encoding a decarboxylase enzyme capable of decarboxylating 4 hydroxyphenylpyruvaic acid (HPP) to produce 4 hydroxyphenylacetaldehyde, and a second polynucleotide sequence encoding a hydroxylase enzyme, wherein the hydroxylase has an amino acid sequence having at least 90% identity to the amino acid sequence as set forth in SEQ ID NO: 2, and the medium comprises glucose; and collecting product hydroxytyrosol from one or both of the host microorganism and the culture medium.

### RELATED APPLICATION

This application claims the benefit under 35 U.S.C. § 119(e) to U.S. Provisional Application No. 63/305,657 filed on Feb. 1, 2022 and entitled “METHODS OF PRODUCING HYDROXYTYROSOL,” the entire contents of which are incorporated herein by reference.

### FIELD OF INVENTION

The field of the invention relates to methods and processes useful in the production of tyrosol and hydroxytyrosol, as well as related compositions.

### REFERENCE TO AN ELECTRONIC SEQUENCE LISTING

The contents of the electronic sequence listing (C149770078W000-SEQ-ZJG.xml; Size: 32,464 bytes; and Date of Creation: Jan. 26, 2023) is herein incorporated by reference in its entirety.

### BACKGROUND

Tyrosol is a phenolic molecule with natural antioxidant properties. It has a chemical name of 4-(2-Hydroxyethyl)phenol, a molecular formula of C₈H₁₀O₂, a molecular weight of 138.164, a CAS number of 501-94-0, and the following structural formula:

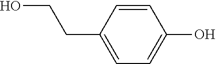

Tyrosol is derived from olive oil and may be chemically synthesized from phenylethanol. It can protect cells from oxidative damage, and is a phenolic compound with important industrial significance. Tyrosol and its derivatives serve as precursors for synthesizing various organic compounds and can also be used in pharmaceutical preparations. Hydroxytyrosol, a derivative of tyrosol, is characterized by robust antioxidation properties and a variety of physiological and medical functions. Hydroxytyrosol has a more potent antioxidation effect than tyrosol, can be used in the synthesis of many polymers, is known to be non-toxic and widely used in biomedicine, functional foods, and other industries, and is useful in the prevention of cardiovascular diseases, osteopenia, and other diseases.

It has a chemical name of 4-(2-Hydroxyethyl)-1,2-benzenediol, a molecular formula of C₈H₁₀O₃, a molecular weight of 154.16, a CAS number of 10597-60-1, and the following structural formula:

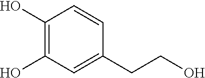 [SMILES: OCCc1ccc(O)c(O)c1]

Hydroxytyrosol is typically obtained by extraction from olive leaves, which has the disadvantages of high cost and occupation of a large amount of arable land.

Organic synthetic methods for manufacturing tyrosol and hydroxytyrosol suffer from low yields or require expensive raw materials. The biosynthesis of hydroxytyrosol in genetically recombinant microorganisms has been reported in patent documents, for example U.S. Pat. Appl. Publ. No. 2010/0068775 titled “Novel genes for the fermentative production of hydroxytyrosol”, and U.S. Pat. Appl. Publ. No. 2021/0254081 titled “Yeast producing tyrosol and hydroxytyrosol, and construction methods thereof”. Both references teach the use of enzyme HpaBC, a 4-phenylacetate hydroxylase, to convert tyrosol to hydroxytyrosol. Unfortunately, the enzyme is also characterized by activity on tyrosine, leading to the production of unwanted side product L-DOPA.

### SUMMARY

Provided herein, in some aspects, are methods and microorganisms for producing one or more of tyrosol and hydroxytyrosol.

In one aspect, provided herein is a method of producing hydroxytyrosol. The method includes: culturing a recombinant host microorganism in a medium, wherein: the host microorganism has been transformed with: a first polynucleotide sequence encoding a decarboxylase enzyme capable of decarboxylating 4-hydroxyphenylpyruvaic acid (HPP) to produce 4-hydroxyphenylacetaldehyde, and a second polynucleotide sequence encoding a hydroxylase enzyme, wherein the hydroxylase has an amino acid sequence having at least 90% identity to the amino acid sequence as set forth in SEQ ID NO: 2, and the medium comprises glucose; and collecting product hydroxytyrosol from one or both of the host microorganism and the culture medium. In one embodiment, the hydroxylase has an amino acid sequence having at least 95% identity to the amino acid sequence as set forth in SEQ ID NO: 2. In another embodiment, the hydroxylase has an amino acid sequence with at least 99% identity to the amino acid sequence as set forth in SEQ ID NO: 2. In a further embodiment, the hydroxylase has the amino acid sequence as set forth in SEQ ID NO: 2. In one embodiment, the decarboxylase has an amino acid sequence with at least 95% identity to the amino acid sequence as set forth in SEQ ID NO: 4. In another embodiment, the decarboxylase has an amino acid sequence having at least 99% identity to the amino acid sequence as set forth in SEQ ID NO: 4. In a further embodiment, the decarboxylase has the amino acid sequence as set forth in SEQ ID NO: 4. In certain embodiments, the host microorganism has been further transformed to overexpress a DAHP synthase relative to a corresponding parental host cell. In a representative embodiment, the DAHP synthase is AroG*. In a non-limiting embodiment, the host microorganism has been further transformed to overexpress an enzyme relative to a corresponding parental host cell, wherein the catalytic activity of the overexpressed enzyme includes converting chorismate (CHA) to prephenate (PPA) relative to a parental host cell. The enzyme whose catalytic activity includes converting chorismate to prephenate may be TyrA*.

In a further aspect, provided herein is a recombinant host microorganism comprising a metabolic pathway producing hydroxytyrosol, wherein the host microorganism has been transformed with a first polynucleotide sequence encoding a decarboxylase enzyme capable of decarboxylating 4-hydroxyphenylpyruvaic acid (HPP) to produce 4-hydroxyphenylacetaldehyde, and a second polynucleotide sequence encoding a hydroxylase enzyme, wherein the hydroxylase has an amino acid sequence with at least 90% identity to the amino acid sequence as set forth in SEQ ID NO: 2. In a representative embodiment, the hydroxylase has an amino acid sequence having at least 95% identity to the amino acid sequence as set forth in SEQ ID NO: 2. In another embodiment, the hydroxylase has an amino acid sequence having at least 99% identity to the amino acid sequence as set forth SEQ ID NO: 2. In a further embodiment, the hydroxylase has the amino acid sequence of SEQ ID NO: 2. In a non-limiting embodiment, the decarboxylase has an amino acid sequence having at least 95% identity to the amino acid sequence as set forth in SEQ ID NO: 4. In a further embodiment, the decarboxylase has an amino acid sequence having at least 99% identity to the amino acid sequence as set forth in SEQ ID NO: 4. In an additional embodiment, the decarboxylase has the amino acid sequence of SEQ ID NO: 4. In another embodiment, the microorganism has been further transformed to overexpress a DAHP synthase relative to a corresponding parental host cell. The DAHP synthase may AroG*. In a further embodiment, the recombinant host microorganism has been further transformed to overexpress an enzyme relative to a corresponding parental host cell, wherein the catalytic activity of the overexpressed enzyme includes converting chorismate (CHA) to prephenate (PPA) relative to a parental host cell. The enzyme whose catalytic activity includes converting chorismate to prephenate may be TyrA*. Non-limiting examples of host organisms include those from the genera Escherichia, Bacillus, Corynebacterium, Saccharomyces, and Yarrowia. In one embodiment, the host microorganism is Escherichia coli.

While the disclosure is susceptible to various modifications and alternative forms, specific embodiments thereof are shown by way of example in the drawing and will herein be described in detail. It should be understood, however, that the drawings and detailed description presented herein are not intended to limit the disclosure to the particular embodiments disclosed, but on the contrary, the intention is to cover all modifications, equivalents, and alternatives falling within the spirit and scope of the present disclosure as defined by the appended claims.

Other features and advantages of this invention will become apparent in the following detailed description of preferred embodiments of this invention, taken with reference to the accompanying drawings.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 illustrates the endogenous shikimate pathway in the context of tyrosine biosynthesis in E. coli.

FIG. 2 illustrates are steps of a novel tyrosol and hydroxytyrosol biosynthetic pathway.

FIG. 3 is a schematic illustration of tyrosine-producing plasmid aroG*-tyrA*-pUVAP.

FIG. 4 is a schematic illustration of hydroxytyrosol-producing plasmid aroG*-tyrA*-PpDKC4-Pp3H03-SeFR1-pUVAP.

FIG. 5 includes chromatograms from the HPLC analysis of broth samples from fermentations carried out in 5 ml shaking tubes.

FIG. 6 includes a table reporting product tyrosol and hydroxytyrosol titers from the broths of experiments that were carried in 2-liter fermenters.

### DEFINITIONS

As used herein, the singular forms “a,” “an” and “the” include plural references unless the content clearly dictates otherwise.

To the extent that the term “include,” “have,” or the like is used in the description or the claims, such term is intended to be inclusive in a manner similar to the term “comprise” as “comprise” is interpreted when employed as a transitional word in a claim.

The word “exemplary” is used herein to mean serving as an example, instance, or illustration. Any embodiment described herein as “exemplary” is not necessarily to be construed as preferred or advantageous over other embodiments.

“Cellular system” is any cells that provide for the expression of proteins. It includes bacteria, yeast, plant cells and animal cells. It includes both prokaryotic and eukaryotic cells. It also includes the in vitro expression of proteins based on cellular components, such as ribosomes.

“Coding sequence” is to be given its ordinary and customary meaning to a person of ordinary skill in the art and is used without limitation to refer to a DNA sequence that encodes a specific amino acid sequence.

“Growing”, “cultivating”, or “culturing” a cellular system includes providing an appropriate liquid or solid medium that would allow cells, for example a population of microbial cells, to multiply and divide. It also includes providing resources so that cells or cellular components can translate and make recombinant proteins. The culturing may be carried out using conventional fermentation equipment suitable for such purpose (e.g., shake flasks, fermentation tanks, and bioreactors).

The term “complementary” is to be given its ordinary and customary meaning to a person of ordinary skill in the art and is used without limitation to describe the relationship between nucleotide bases that are capable to hybridizing to one another. For example, with respect to DNA, adenosine is complementary to thymine and cytosine is complementary to guanine. Accordingly, the subjection technology also includes isolated nucleic acid fragments that are complementary to the complete sequences as reported in the accompanying Sequence Listing as well as those substantially similar nucleic acid sequences.

The terms “nucleic acid” and “nucleotide” are to be given their respective ordinary and customary meanings to a person of ordinary skill in the art and are used without limitation to refer to deoxyribonucleotides or ribonucleotides and polymers thereof in either single- or double-stranded form. Unless specifically limited, the term encompasses nucleic acids containing known analogues of natural nucleotides that have similar binding properties as the reference nucleic acid and are metabolized in a manner similar to naturally-occurring nucleotides. In any one embodiments provided herein, a particular nucleic acid sequence can also encompass conservatively modified or degenerate variants thereof (e.g., degenerate codon substitutions) and complementary sequences, as well as the sequence explicitly indicated.

The term “isolated” is to be given its ordinary and customary meaning to a person of ordinary skill in the art, and when used in the context of an isolated nucleic acid or an isolated polypeptide, is used without limitation to refer to a nucleic acid or polypeptide that, by the hand of man, exists apart from its native environment and is therefore not a product of nature. An isolated nucleic acid or polypeptide can exist in a purified form or can exist in a non-native environment such as, for example, in a transgenic host cell.

The terms “incubating” and “incubation” as used herein means a process of mixing two or more chemical or biological entities (such as a chemical compound and an enzyme) and allowing them to interact under conditions favorable for producing one or more of tyrosol and hydroxytyrosol.

The term “degenerate variant” refers to a nucleic acid sequence having a residue sequence that differs from a reference nucleic acid sequence by one or more degenerate codon substitutions. Degenerate codon substitutions can be achieved by generating sequences in which the third position of one or more selected (or all) codons is substituted with mixed base and/or deoxyinosine residues. A nucleic acid sequence and all of its degenerate variants will express the same amino acid or polypeptide.

The terms “polypeptide,” “protein,’ and “peptide” are to be given their respective ordinary’ and customary meanings to a person of ordinary skill in the art; the three terms are sometimes used interchangeably and are used without limitation to refer to a polymer of amino acids, or amino acid analogs, regardless of its size or function. Although “protein” is often used in reference to relatively large polypeptides, and “peptide” is often used in reference to small polypeptides, usage of these terms in the art overlaps and varies. The term ‘polypeptide” as used herein refers to peptides, polypeptides, and proteins, unless otherwise noted. The terms “protein,” “polypeptide,” and “peptide” are used interchangeably herein when referring to a polynucleotide product. Thus, exemplary polypeptides include polynucleotide products, naturally occurring proteins, homologs, orthologs, paralogs, fragments and other equivalents, variants, and analogs of the foregoing.

The terms “polypeptide fragment” and “fragment,” when used in reference to a reference polypeptide, are to be given their ordinary and customary meanings to a person of ordinary skill in the art and are used without limitation to refer to a polypeptide in which amino acid residues are deleted as compared to the reference polypeptide itself, but where the remaining amino acid sequence is usually identical to the corresponding positions in the reference polypeptide. Such deletions can occur at the amino-terminus or carboxy-terminus of the reference polypeptide, or alternatively both.

The term “functional fragment” of a polypeptide or protein refers to a peptide fragment that is a portion of the full-length polypeptide or protein, and has substantially the same biological activity, or carries out substantially the same function as the full-length polypeptide or protein (e.g., carrying out the same enzymatic reaction).

The terms “variant polypeptide,” “modified amino acid sequence” or “modified polypeptide,” which are used interchangeably, refer to an amino acid sequence that is different from the reference polypeptide by one or more amino acids, e.g., by one or more amino acid substitutions, deletions, and/or additions. In an aspect, a variant is a “functional variant” which retains some or all of the ability of the reference polypeptide. In any one embodiment, the AghSHC1 polypeptide may be a functional variant.

The term “functional variant” further includes conservatively substituted variants. The term “conservatively substituted variant” refers to a peptide having an amino acid sequence that differs from a reference peptide by one or more conservative amino acid substitutions and maintains some or all of the activity of the reference peptide. A “conservative amino acid substitution” is a substitution of an amino acid residue with a functionally similar residue. Examples of conservative substitutions include the substitution of one non-polar (hydrophobic) residue such as isoleucine, valine, leucine or methionine for another: the substitution of one charged or polar (hydrophilic) residue for another such as between arginine and lysine, between glutamine and asparagine, between threonine and serine; the substitution of one basic residue such as lysine or arginine for another, or the substitution of one acidic residue, such as aspartic acid or glutamic acid for another; or the substitution of one aromatic residue, such as phenylalanine, tyrosine, or tryptophan for another. Such substitutions are expected to have little or no effect on the apparent molecular weight or isoelectric point of the protein or polypeptide. The phrase “conservatively substituted variant” also includes peptides wherein a residue is replaced with a chemically-derivatized residue, provided that the resulting peptide maintains some or all of the activity of the reference peptide as described herein.

The term “variant,” in connection with the polypeptides of the subject technology, further includes a functionally active polypeptide having an amino acid sequence at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, and even 100% identical to the amino acid sequence of a reference polypeptide. In any one embodiment, the variant polypeptide may be a variant with any one of the foregoing percentage identities. In instances where the reference polypeptide is an enzyme, preferably the variant polypeptide is functional in the conversion catalyzed by the reference polypeptide.

The term “homologous” in all its grammatical forms and spelling variations refers to the relationship between polynucleotides or polypeptides that possess a “common evolutionary origin,” including polynucleotides or polypeptides from super families and homologous polynucleotides or proteins from different species (Reeck et al., CELL 50:667, 1987). Such polynucleotides or polypeptides have sequence homology, as reflected by their sequence similarity, whether in terms of percent identity or the presence of specific amino acids or motifs at conserved positions. For example, two homologous polypeptides can have amino acid sequences that are at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 900 at least 91%, at least 92%, at least 93%, at least 94%, at least 95%, at least 96% r, at least 97%, at least 98%, at least 99%, and even 100% identical.

“Suitable regulatory sequences” is to be given its ordinary and customary meaning to a person of ordinary skill in the art and is used without limitation to refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, and polyadenylation recognition sequences.

“Promoter” is to be given its ordinary and customary meaning to a person of ordinary skill in the art and is used without limitation to refer to a DNA sequence capable of controlling the expression of a coding sequence or functional RNA. In general, a coding sequence is located 3′ to a promoter sequence. Promoters may be derived in their entirety from a native gene or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. Promoters, which cause a gene to be expressed in most cell types at most times, are commonly referred to as “constitutive promoters.” It is further recognized that since in most cases the exact boundaries of regulatory sequences have not been completely defined, DNA fragments of different lengths may have identical promoter activity.

The term “operably linked” refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

The term “expression” as used herein, is to be given its ordinary and customary meaning to a person of ordinary skill in the art and is used without limitation to refer to the transcription and stable accumulation of sense (mRNA) or antisense RNA derived from the nucleic acid fragment of the subject technology or production of a gene product in transgenic, transformed or recombinant organisms.

“Transformation” is to be given its ordinary and customary meaning to a person of ordinary skill in the art and is used without limitation to refer to the transfer of a polynucleotide into a target cell. The transferred polynucleotide can be incorporated into the genome or chromosomal DNA of a target cell, resulting in genetically stable inheritance, or it can replicate independent of the host chromosomal. Host organisms containing the transformed nucleic acid fragments are referred to as “transgenic” or “transformed” or “recombinant”.

The terms “transformed,” “transgenic,” and “recombinant,” when used herein in connection with host cells, are to be given their respective ordinary and customary meanings to a person of ordinary skill in the art and are used without limitation to refer to a cell of a host organism, such as a plant or microbial cell, into which a heterologous nucleic acid molecule has been introduced. The nucleic acid molecule can be stably integrated into the genome of the host cell, or the nucleic acid molecule can be present as an extrachromosomal molecule. Such an extrachromosomal molecule can be auto-replicating. Transformed cells, tissues, or subjects are understood to encompass not only the end product of a transformation process, but also transgenic progeny thereof.

The terms “recombinant,” “heterologous,” and “exogenous,” when used herein in connection with polynucleotides, are to be given their ordinary and customary meanings to a person of ordinary skill in the art and are used without limitation to refer to a polynucleotide (e.g., a DNA sequence or a gene) that originates from a source foreign to the particular host cell or, if from the same source, is modified from its original form. Thus, a heterologous gene in a host cell includes a gene that is endogenous to the particular host cell but has been modified through, for example, the use of site-directed mutagenesis or other recombinant techniques. The terms also include non-naturally occurring multiple copies of a naturally occurring DNA sequence. Thus, the terms refer to a DNA segment that is foreign or heterologous to the cell, or homologous to the cell but in a position or form within the host cell in which the element is not ordinarily found.

Similarly, the terms “recombinant,” “heterologous,” and “exogenous,” when used herein in connection with a polypeptide or amino acid sequence, means a polypeptide or amino acid sequence that originates from a source foreign to the particular host cell or, if from the same source, is modified from its original form. Thus, recombinant DNA segments can be expressed in a host cell to produce a recombinant polypeptide.

“Protein Expression” refers to protein production that occurs after gene expression. It consists of the stages after DNA has been transcribed to messenger RNA (mRNA). The mRNA is then translated into polypeptide chains, which are ultimately folded into proteins. DNA is present in the cells through transfection—a process of deliberately introducing nucleic acids into cells. The term is often used for non-viral methods in eukaryotic cells. It may also refer to other methods and cell types, although other terms are preferred: “transformation” is more often used to describe non-viral DNA transfer in bacteria, non-animal eukaryotic cells, including plant cells. In animal cells, transfection is the preferred term as transformation is also used to refer to progression to a cancerous state (carcinogenesis) in these cells. Transduction is often used to describe virus-mediated DNA transfer. Transformation, transduction, and viral infection are included under the definition of transfection for this application.

The terms “plasmid,” “vector,” and “cassette” are to be given their respective ordinary and customary meanings to a person of ordinary skill in the art and are used without limitation to refer to an extra chromosomal element often carrying genes which are not part of the central metabolism of the cell, and usually in the form of circular double-stranded DNA molecules. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell. “Transformation cassette” refers to a specific vector containing a foreign gene and having elements in addition to the foreign gene that facilitate transformation of a particular host cell. “Expression cassette” refers to a specific vector containing a foreign gene and having elements in addition to the foreign gene that allow for enhanced expression of that gene in a foreign host.

As used herein “sequence identity” refers to the extent to which two optimally aligned polynucleotide or peptide sequences are invariant throughout a window of alignment of components, e.g., nucleotides or amino acids. An “identity fraction” for aligned segments of a test sequence and a reference sequence is the number of identical components which are shared by the two aligned sequences divided by the total number of components in reference sequence segment, i.e., the entire reference sequence or a smaller defined part of the reference sequence.

As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned (with appropriate nucleotide insertions, deletions, or gaps totaling less than 20 percent of the reference sequence over the window of comparison). Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and preferably by computerized implementations of these algorithms such as GAP, BESTFIT, FASTA, and TFASTA available as part of the GCG® Wisconsin Package® (Accelrys Inc., Burlington, MA). An “identity fraction” for aligned segments of a test sequence and a reference sequence is the number of identical components which are shared by the two aligned sequences divided by the total number of components in the reference sequence segment, i.e., the entire reference sequence or a smaller defined part of the reference sequence. Percent sequence identity is represented as the identity fraction multiplied by 100. The comparison of one or more polynucleotide sequences may be to a full-length polynucleotide sequence or a portion thereof, or to a longer polynucleotide sequence. For purposes of this invention “percent identity” may also be determined using BLASTX version 2.0 for translated nucleotide sequences and BLASTN version 2.0 for polynucleotide sequences.

The percent of sequence identity is preferably determined using the “Best Fit” or “Gap” program of the Sequence Analysis Software Package™ (Version 10: Genetics Computer Group, Inc., Madison, WI). “Gap” utilizes the algorithm of Needleman and Wunsch (Needleman and Wunsch, JOURNAL OF MOLECULAR BIOLOGY 48:443-453, 1970) to find the alignment of two sequences that maximizes the number of matches and minimizes the number of gaps. “BestFit” performs an optimal alignment of the best segment of similarity between two sequences and inserts gaps to maximize the number of matches using the local homology algorithm of Smith and Waterman (Smith and Waterman, ADVANCES IN APPLIED MATHEMATICS, 2:482-489, 1981. Smith et al., NUCLEIC ACIDS RESEARCH 11:2205-2220, 1983). The percent identity is most preferably determined using the “Best Fit” program.

Useful methods for determining sequence identity are also disclosed in the Basic Local Alignment Search Tool (BLAST) programs which are publicly available from National Center Biotechnology Information (NCBI) at the National Library of Medicine, National Institute of Health, Bethesda, Md. 20894; see BLAST Manual, Altschul et al., NCBI, NLM, NIH; Altschul et al., J. MOL. BIOL. 215:403-410 (1990); version 2.0 or higher of BLAST programs allows the introduction of gaps (deletions and insertions) into alignments; for peptide sequence BLASTX can be used to determine sequence identity; and, for polynucleotide sequence BLASTN can be used to determine sequence identity.

As used herein, the term “substantial percent sequence identity” refers to a percent sequence identity of at least about 70% sequence identity, at least about 80% sequence identity, at least about 85% identity, at least about 90% sequence identity, or even greater sequence identity, such as about 98% or about 99% sequence identity. Thus, one embodiment of the invention is a polynucleotide molecule that has at least about 70% sequence identity, at least about 80% sequence identity, at least about 85% identity, at least about 90% sequence identity, or even greater sequence identity, such as about 98% or about 99% sequence identity with a polynucleotide sequence described herein. Polynucleotide molecules that have the activity genes of the current invention are useful in the production of tyrosol and hydroxytyrosol as provided herein and have a substantial percent sequence identity to the polynucleotide sequences provided herein and are encompassed within the scope of this invention.

Identity is the fraction of amino acids that are the same between a pair of sequences after an alignment of the sequences (which can be done using only sequence information or structural information or some other information, but usually it is based on sequence information alone), and similarity is the score assigned based on an alignment using some similarity matrix. The similarity index can be any one of the following BLOSUM62, PAM250, or GONNET, or any matrix used by one skilled in the art for the sequence alignment of proteins.

Identity is the degree of correspondence between two sub-sequences (no gaps between the sequences). An identity of 25% or higher implies similarity of function, while 18-25% implies similarity of structure or function. Keep in mind that two completely unrelated or random sequences (that are greater than 100 residues) can have higher than 20% identity. Similarity is the degree of resemblance between two sequences when they are compared. This is dependent on their identity.

### DETAILED DESCRIPTION

Provided herein, in one aspect, are recombinant microorganisms capable of producing tyrosol. In a second aspect, there are provided recombinant microorganisms capable of producing hydroxytyrosol. In a third aspect, there is provided a method for constructing recombinant microorganisms capable of producing tyrosol. In a fourth aspect, provided herein is a method for constructing recombinant microorganisms capable of producing hydroxytyrosol. In a fifth aspect, there is provided a method whereby the recombinant microorganisms capable of producing tyrosol are used in the production of tyrosol. In a sixth aspect, there is provided a method whereby the recombinant microorganisms capable of producing hydroxytyrosol are used in the production of hydroxytyrosol.

The recombinant microorganisms may be obtained by genetic engineering of any host species that is suitable to the production of tyrosol and hydroxytyrosol. Non-limiting examples include bacteria of the genera Escherichia, Bacillus, Corynebacterium, and fungi of the genera Saccharomyces and Yarrowia.

A first example of recombinant bacterial strain for the heterologous biosynthesis of tyrosol is developed by constructing a biosynthetic pathway in a host bacterium species. A first exogenous coding sequence encoding a decarboxylase capable of decarboxylating 4-hydroxyphenylpyruvaic acid (HPP) is introduced, thereby enabling the bacterium to produce 4-hydroxyphenylacetaldehyde (HAA or 4-HAA) which may be converted to tyrosol by an endogenous alcohol dehydrogenase, for example ADH. A second bacterial strain for heterologous biosynthesis of hydroxytyrosol is constructed by further transforming the first recombinant bacterium with a second exogenous coding sequence encoding a hydroxylase capable of selectively hydroxylating tyrosol, to yield hydroxytyrosol.

Accordingly, some embodiments of the present disclosure relate to a recombinant bacterium (e.g., recombinant E. coli) comprising one or more heterologous nucleic acid molecules that includes a polynucleotide encoding either the decarboxylase, the hydroxylase, or both. In some embodiments, the bacteria (e.g., E. coli) are transformed with nucleic acid molecules (e.g., plasmids) comprising one or both of a first polynucleotide encoding the decarboxylase and the hydroxylase.

### Coding Nucleic Acid Sequences

To form a desired recombinant microorganism, provided herein are nucleic acid sequences that code for the hydroxylase enzyme and which can be applied to perform the required genetic engineering manipulations. Also provided are nucleic acid sequences with a substantial percent sequence identity to the sequences specifically disclosed herein. For example, aspects of the present invention encompass a nucleic acid sequence with at least at least 70% identity to SEQ ID NO. 1, at least 75% identity to SEQ ID NO. 1, at least 80% identity to SEQ ID NO. 1, at least 85% identity to SEQ ID NO. 1, at least 90% identity to SEQ ID NO. 1, at least 95% identity to SEQ ID NO. 1, or at least 99% identity to SEQ ID NO. 1. In some embodiments, the nucleic acid sequence used to encode a hydroxylase useful for the present invention can have a nucleic acid sequence identical to SEQ ID NO. 1.

In some embodiments, the hydroxylase enzyme described herein comprises an amino acid sequence that is at least 70% (e.g., at least 70%, at least 75%, at least 80%, at least 85%, at least 90%, at least 95%, at least 98%, or at least 99%) identical to the amino acid sequence of Ph3H03, i.e., a newly characterized hydroxylase enzyme endogenous to Gordonia bronchialis which has the amino acid sequence of SEQ ID NO: 2. In some embodiments, the amino acid sequence of the hydroxylase enzyme is identical to Ph3H03. As used herein, the term “Ph3H03” refers to a full-length enzyme or a fragment or variants thereof retaining the ability to hydroxylate tyrosol, to form hydroxytyrosol.

Also provided herein are nucleic acid sequences that code for the decarboxylase enzyme and which can likewise be applied to perform the required genetic engineering manipulations. Also provided are nucleic acid sequences with a substantial degree of identity to the sequences specifically disclosed herein. For example, embodiments of the present invention encompass a nucleic acid sequence with at least 70% identity to SEQ ID NO. 3, at least 75% identity to SEQ ID NO. 3, at least 80% identity to SEQ ID NO. 3, at least 85% identity to SEQ ID NO. 3, at least 90% identity to SEQ ID NO. 3, at least 95% identity to SEQ ID NO. 3, or at least 99% identity to SEQ ID NO. 3. In some embodiments, the nucleic acid sequence which are used to encode a decarboxylase useful for the present invention can have a nucleic acid sequence identical to SEQ ID NO. 3.

In some embodiments, the decarboxylase enzyme described herein comprises an amino acid sequence that is at least 70% (e.g., at least 70%, at least 75%, at least 80%, at least 85%, at least 90%, at least 95%, at least 98%, or at least 99%) identical to the amino acid sequence of PpkDC4, i.e., a hydroxylase enzyme having an amino acid sequence as shown in SEQ ID NO: 4. In some embodiments, the amino acid sequence of the hydroxylase enzyme is identical to PpkDC4. As used herein, the term “PpkDC4” refers to a full-length enzyme or a fragment or variants thereof retaining the ability to decarboxylate HPP, to form 4-hydroxyphenylacetaldehyde.

In some embodiments, a polynucleotide(s) encoding one or both of the decarboxylase enzyme and hydroxylase enzyme is operably linked to a promoter. In some embodiments, the promoter is a constitutive promoter (e.g., a constitutive promoter in a bacterium). In some embodiments, the promoter comprises a mutated promoter (e.g., a bacterial lacUV5 promoter). In some embodiments, a polynucleotide encoding one or more of the decarboxylase enzyme and hydroxylase enzyme is operably linked to a transcription terminator. In some embodiments, the transcription is bacteriophage T7 terminator.

### Cell Transformation

The nucleic acid molecule encoding one or both of the decarboxylase and hydrolase enzyme may be inserted into a host species, e.g., a bacterium, in the form of a vector (e.g., an expression vector). In a representative embodiment, a Ph3H03- and a PpkDC4-encoding sequences are inserted into a pUVAP plasmid, to construct a pUVAP-Ph3H03-PpkDC4 expression vector. Typically, the expression vector includes those genetic elements for expression of recombinant polypeptide(s) in host cells. The elements for transcription and translation in the host cell can include a promoter, a coding region for the protein complex, and a transcriptional terminator.

A person of ordinary skill in the art will be aware of the molecular biology techniques available for the preparation of expression vectors. The polynucleotide used for incorporation into the expression vector of the subject technology, as described above, can be prepared by routine techniques such as polymerase chain reaction (PCR).

Several molecular biology techniques can be developed to operably link DNA to vectors via complementary cohesive termini. In one embodiment, complementary homopolymer tracts can be added to the nucleic acid molecule to be inserted into the vector DNA. The vector and nucleic acid molecule are then joined by hydrogen bonding between the complementary homopolymeric tails to form recombinant DNA molecules.

In some embodiments, synthetic linkers containing one or more restriction sites provide are used to operably link the polynucleotide(s) of the subject technology to the expression vector. In an embodiment, the polynucleotide is generated by restriction endonuclease digestion. In an embodiment, the nucleic acid molecule is treated with bacteriophage T4 DNA polymerase or E. coli DNA polymerase I, enzymes that remove protruding, 3′-single-stranded termini with their 3′-5′-exonucleolytic activities and fill in recessed 3′-ends with their polymerizing activities, thereby generating blunt ended DNA segments. The blunt-ended segments are then incubated with a large molar excess of linker molecules in the presence of an enzyme that can catalyze the ligation of blunt-ended DNA molecules, such as bacteriophage T4 DNA ligase. Thus, the product of the reaction is a polynucleotide carrying polymeric linker sequences at its ends. These polynucleotides are then cleaved with the appropriate restriction enzyme and ligated to an expression vector that has been cleaved with an enzyme that produces termini compatible with those of the polynucleotide.

Alternatively, a vector having ligation-independent cloning (LIC) sites can be employed. The required PCR amplified polynucleotide can then be cloned into the LIC vector without restriction digest or ligation (Aslanidis and de Jong, NUCL. ACID. RES. 18 6069-74, (1990), Haun, et al, BIOTECHNIQUES 13, 515-18 (1992), which is incorporated herein by reference to the extent it is consistent herewith).

In some embodiments, to isolate and/or modify the polynucleotide(s) of interest for insertion into the chosen plasmid, it is suitable to use PCR. Appropriate primers for use in PCR preparation of the sequence can be designed to isolate the required coding region of the nucleic acid molecule, add restriction endonuclease or LIC sites, place the coding region in the desired reading frame.

In some embodiments, a polynucleotide for incorporation into an expression vector of the subject technology is prepared using PCR using appropriate oligonucleotide primers. The coding region is amplified, whilst the primers themselves become incorporated into the amplified sequence product. In some embodiments, the amplification primers contain restriction endonuclease recognition sites, which allow the amplified sequence product to be cloned into an appropriate vector.

The expression vectors can be introduced into the bacterial cells (e.g., E. coli) by conventional transformation or transfection techniques. Transformation of appropriate cells with an expression vector of the subject technology is accomplished by methods known in the art and typically depends on both the type of vector and cell. Suitable techniques include calcium phosphate or calcium chloride co-precipitation, DEAE-dextran mediated transfection, lipofection, chemoporation or electroporation.

Successfully transformed cells, that is, those cells containing the expression vector, can be identified by techniques well known in the art. For example, bacterial cells (e.g., E. coli) transfected with an expression vector of the subject technology can be cultured to produce polypeptides described herein. Cells can be examined for the presence of the expression vector DNA by techniques well known in the art. The host cells can contain a single copy of the expression vector described previously, or alternatively, multiple copies of the expression vector.

In some embodiments, the nucleic acid molecule (e.g., vector) inserted in the bacterial cell further comprises a polynucleotide encoding a selection marker. A “selection marker” is a gene introduced into a cell, especially cells in culture, that confers a trait suitable for artificial selection. In some embodiments, a selectable marker is a gene that confers resistance to a drug to eukaryotic cells, including but not limited to paromomycin, puromycin, hygromycin, G418, neomycin, or bleomycin. In some embodiments, a selectable marker is a gene that confers resistance to paromomycin.

### Biosynthesis of Tyrosol and Hydroxytyrosol

In another aspect, provided herein are methods for the de novo synthesis of one or more of tyrosol and hydroxytyrosol using the recombinant host species of the present disclosure (e.g., recombinant E. coli). An exemplary such method includes culturing recombinant bacteria (e.g., recombinant E. coli cells) under conditions that that allow expression of exogenous genes relating to the production of tyrosol and hydroxytyrosol. In certain embodiments, the method further comprises isolating one or more of product tyrosol and hydroxytyrosol that is present in one or both of the bacterial cells and culturing medium.

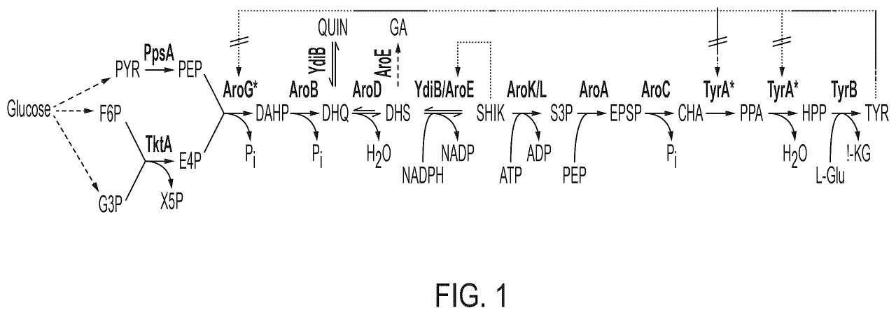

The tyrosol and hydroxytyrosol may be made from an HPP substrate. Typically, the HPP is a downstream intermediate of the shikimate pathway, which provides the precursors to tyrosine and other aromatic amino acids by de novo synthesis from glucose. FIG. 1 illustrates the endogenous shikimate pathway in the context of tyrosine biosynthesis in E. coli. Metabolic engineering of the shikimate pathway may be undertaken with the goal of inducing an over-production of the HPP available for tyrosol production. This may be accomplished by overexpressing one or more of the shikimate pathway enzymes that are endogenous to a given bacterial species. Illustrative embodiments of this strategy as applied to E. coli involve the overexpression of one or more of the enzymes of FIG. 1. In one representative example, DAHP synthase AroG* is expressed at levels exceeding those of the wild-type bacterium. An analogous approach entails the overexpression of TyrA*, an E. coli enzyme whose catalytic activity includes the conversion of chorismate (CHA) to prephenate (PPA) which in turn undergoes decarboxylation, to yield HPP. In some embodiments, the E. coli cell is engineered to overexpress both enzymes TyrA* and AroG*.

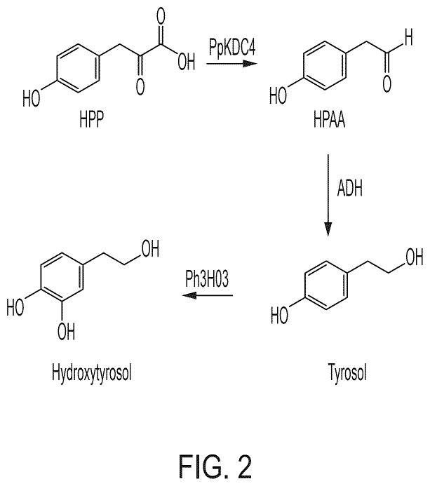

Illustrated in FIG. 2 are subsequent steps of a de novo biosynthetic pathway according to an aspect of the present invention. HPP is decarboxylated to form 4-hydroxyphenylacetaldehyde (4-HPAA) which in turn undergoes reduction at its aldehydic moiety, to yield tyrosol. In a further step, the tyrosol is hydroxylated to produce hydroxytyrosol. In one exemplary embodiment, the decarboxylation of HPP is catalyzed by PpKDC4, a phenylpyruvate decarboxylase from Pichia pastoris. Without being bound to any particular theory, it is believed that an endogenous enzyme belonging to the family of alcohol dehydrogenases (ADH) catalyzes the formation of tyrosol.

As anticipated above, traditional methods rely on hydroxylase HpaBC to convert tyrosol to hydroxytyrosol. The present invention is based, at least in part, on the discovery that Ph3H03, an enzyme from Gordonia bronchialis, is characterized by high activity and specificity in the hydroxylation of tyrosol, to yield hydroxytyrosol. Without wishing to be bound to any particular theory, it is believed that Ph3H03 acts in conjunction with flavin reductase SeFR1 to convert tyrosol into hydroxytyrosol with high activity. Importantly, and unlike HpaBC, Ph3H03 was found to be inactive towards tyrosine, and E. coli strain expressing Ph3H03 were not found to yield significant amounts of undesired L-DOPA side product.

A “submerged liquid culture”, as defined herein, is a cell culture in which the cells are suspended, or significantly suspended, in a liquid medium containing nutrients required for maintaining the viability of the cells. The culture is generally agitated at a sufficient rate to ensure distribution of the cells throughout the medium. The agitation rate is typically also selected to prevent formation of concentration gradients of nutrients.

In a “batch process” or “batch fermentation”, all the necessary culture and media components, with the exception of oxygen for aerobic processes, are placed in a reactor at the start of the operation and the fermentation is allowed to proceed until completion, at which point the product is withdrawn from the reactor.

In a “fed-batch process” or “fed-batch fermentation”, the culture is fed continuously or sequentially with one or more media components without the removal of the culture fluid.

In a “continuous process” or “continuous fermentation”, fresh medium is supplied and culture fluid is removed continuously at volumetrically equal, or substantially equal, rates to maintain the culture at a steady growth rate. In reference to continuous processes, “steady state” refers to a state in which the concentration of reactants does not vary appreciably, and “quasi-steady state” refers to a state in which, subsequent to the initiation of the reaction, the concentration of reactants fluctuates within a range consistent with normal operation of the continuous hydrolysis process. Continuous fermentation process may also be referred to as CSTR (continuous stirred-tank reactor) fermentations. One example of a continuous fermentation process is a chemostat, in which the growth rate of the microorganism is controlled by the supply of one limiting nutrient in the medium.

In the fermentation processes of the present invention, the recombinant host cell may be first cultured in a batch fermentation typically containing a non-inducing carbon source. Upon completion of the batch fermentation, which is typically identified by the depletion of essentially all of the available carbon source, for example, when the concentration of the carbon source in the culture filtrate is no more than 1 g/L, the recombinant host cell is cultured in a fed-batch, continuous or combined fed-batch and continuous submerged liquid culture.

Fed-batch and continuous processes are typically carried out in one or more bioreactors. Typical bioreactors used for cell culture fermentation processes include, but are not limited to, mechanically agitated vessels or those with other means of agitation (such as air injection). Bioreactors may be temperature and pH-controlled. Typically, there are means provided to clean the reactor in place. Means may also be provided to sanitize or sterilize the bioreactor prior to introduction of the target organism so as to minimize or prevent competition for carbon sources from other organisms. Bioreactors may be constructed from many materials, but most often are of glass or stainless steel. Provisions are generally made for sampling (in a manner that prevents or minimizes the introduction of undesirable competing organisms). Means to obtain other measurements are often provided (e.g., ports and probes to measure dissolved oxygen concentration or concentration of other solutes such as ammonium ions). The practice of the invention is not limited by the choice of bioreactor(s).

In the fermentation processes of the present invention, the fed-batch, continuous or combined fed-batch and continuous submerged liquid culture is provided with a feed solution containing a carbon source. In some embodiments, the carbon source consists of one or more carbohydrate. As used herein, the term “carbon source” refers to a carbon-containing substance that provides the major part of the carbon required for growth of, and production of one or more of tyrosol and hydroxytyrosol by a parental or recombinant cell culture. For the purposes herein, a carbon source may be one or more carbohydrate, a non-carbohydrate substance such as a sugar alcohol, organic acid, or alcohol, or combinations thereof. However, for the purposes herein, organic nitrogen sources that may be provided to the cell culture may or may not be considered carbon sources.

In the fermentation process of the present invention, the feed solution may contain one or more additional components, such as nitrogen sources, vitamins, minerals and salts required for growth of the bacterial cell as in known to one of skill in the art. Nitrogen sources may be inorganic and/or organic in nature and include, but are not limited to, one or more amino acids, peptides and proteins, in pure or raw form (e.g., corn steep liquor), any number of protein hydrolysates (peptone, tryptone, casamino acids), yeast extract, ammonia, ammonium hydroxide, ammonium salts, urea, nitrate and combinations thereof. The practice of the fermentation process of the present invention is not limited by the additional components of the feed solution.

The feed solution is provided to the fermentation process at a rate, the feed rate or “carbon addition rate” or “CAR” (measured as g carbon per liter per hour). In an exemplary fermentation process according to the present invention, the feed solution may be provided to a fed-batch culture at a carbon addition rate of from about 0.2 to about 4 g carbon/L culture/h or any rate therebetween, for example 0.2, 0.3, 0.4, 0.6, 0.8, 1.0, 1.2, 1.4, 1.6, 1.8, 2.0, 2.2. 2.5, 3.0, 3.5, and 4.0 g carbon/L culture/h or any rate therebetween. Alternatively, the feed solution may be provided to a continuous culture at a dilution rate of from about 0.001 to 0.1 h−1, or any dilution rate therebetween, for example at about 0.001, 0.005, 0.01, 0.02, 0.03, 0.04, 0.05, 0.06, 0.07, 0.08, 0.09, 0.1 h−1, or any dilution rate therebetween.

The fermentation processes of the present invention may be carried at a temperature from about 20° C. to about 55° C., or any temperature therebetween, for example from about 30° C. to about 45° C., or any temperature therebetween, or from 20, 22, 25, 28, 30, 32, 35, 38, 40, 42, 45, 48, 50° C., 55° C. or any temperature therebetween.

The fermentation processes of the present invention may be carried out at a pH from about 2.5 to 8.5, or any pH therebetween, for example from about pH 3.5 to pH 7.0, or any pH therebetween, for example from about pH 2.5, 3.0, 3.2, 3.5, 3.8, 4.0, 4.2, 4.5, 4.8, 5.0, 5.2, 5.4, 5.5, 5.7, 5.8, 6.0, 6.2, 6.5, 6.8, 7.0, 7.2, 7.5, 7.8, 8.0, 8.5 or any pH therebetween. The pH may be controlled by the addition of a base, such as ammonium or sodium hydroxide, or by the addition of an acid, such as phosphoric acid.

The fermentation processes of the present invention may be carried out over a period of about 1 to 90 days, or any period therebetween, for example between 3 and 30 days, or any amount therebetween, between 3 and 8 days, or any amount therebetween, or from 1, 2, 3, 4, 5, 6, 7, 8, 9, 10, 12, 14, 16, 18, 20, 22, 24, 26, 28, 30, 40, 50, 60, 70, 80, or 90 days, or any amount therebetween.

The fermentation processes of the present invention may be performed in cultures having a volume of at least 0.5 liter, for example from about 0.5 to about 1,000,000 liters, or any amount therebetween, for example, 5 to about 400,000 liters, or any amount therebetween, 20 to about 200,000 liters, or any amount therebetween, or 2,000 to about 200,000 liters, or any amount therebetween, or from about 0.5, 1, 10, 50, 100, 200, 400, 600, 800, 1000, 2000, 4000, 6000, 8000, 10,000, 15,000, 20,000, 25,000, 30,000, 35,000, 40,000, 45,000, 50,000, 55,000, 60,000, 65,000, 70,000, 75,000, 80,000, 85,000, 90,000, 95,000, 100,000, 150,000, 200,000, 300,000, 400,000, 500,000, 750,000 or 1.000,000 liters in volume, or any amount therebetween.

The fermentation processes of the present invention may be performed aerobically, in the presence of oxygen, or anaerobically, in the absence of oxygen. For example, the process may be performed aerobically such that air or oxygen gas is provided to the submerged liquid culture at a superficial gas velocity of from about 0.001 to about 100 cm/s, or any rate therebetween, for example any rate from about 0.01 to about 20 cm/s, or any rate therebetween. An alternative parameter to measure aeration rate that is known to one of skill in the art is vessel volumes per minute (vvm). In the fermentation process of the present invention, air or oxygen gas is provided to the submerged liquid culture at a rate of from about 0.5 to about 5 vvm, or any rate therebetween. Antifoaming agents (either silicone, or non-silicone based) may be added to control excessive foaming during the process as required and as is known to one of skill in the art.

As used herein, the term “specific productivity”, alternatively expressed as “qp”, refers to the rate at which one or more of tyrosol and hydroxytyrosol are produced from a given mass of recombinant host cells. Typically, the specific productivity of a fermentation process is expressed as mg protein per g of host cells per hour (mg protein/g cells/h) and is calculated by measuring the concentration, in mg/L, of one or more of tyrosol and hydroxytyrosol in culture filtrates (culture media from which the recombinant cells have been removed) and dividing by the concentration of cultured cells (in g dry weight per L) in the culture medium and dividing by the total time, in h, since the feed solution was initially provided to the culture. The fermentation processes of the present invention may also be characterized by “maximum productivity” (or “maximum qp”), which is the highest value qp calculated during the course of the fermentation process, or by “average productivity” (or “average qp”), which is the average of all of the values of qp calculated during the course of the fermentation process

As used herein, the terms “equivalent fermentation process” or “equivalent process”, refer to a fermentation process in which a parental bacterial cell is cultured under identical or nearly identical conditions of medium composition, time, cell density, temperature, and pH, as those used to culture an isolated bacterial cell derived from that parental bacterial cell.

After cultivation, products such as hydroxytyrosol may be extracted directly from the liquid medium. In addition, solids such as cells may be removed from the medium by centrifugation or membrane filtration, and the products may be collected and purified by ion-exchange, concentration, distillation, and crystallization methods.

Unless defined otherwise, all technical and scientific terms used herein have the same meaning as commonly understood by one of ordinary skill in the art to which the disclosure belongs. Although any methods and materials similar to or equivalent to those described herein may be used in the practice or testing of the present disclosure, the preferred materials and methods are described below.

### EXAMPLES

The subject technology is further defined in the following Examples. It should be understood that these Examples, while indicating preferred embodiments of the subject technology, are given by way of illustration only. From the above discussion and these Examples, one skilled in the art can ascertain the essential characteristics of the subject technology, and without departing from the spirit and scope thereof, can make various changes and modifications of the subject technology to adapt it to various uses and conditions.

### Example 1: Construction of the Expression Vector

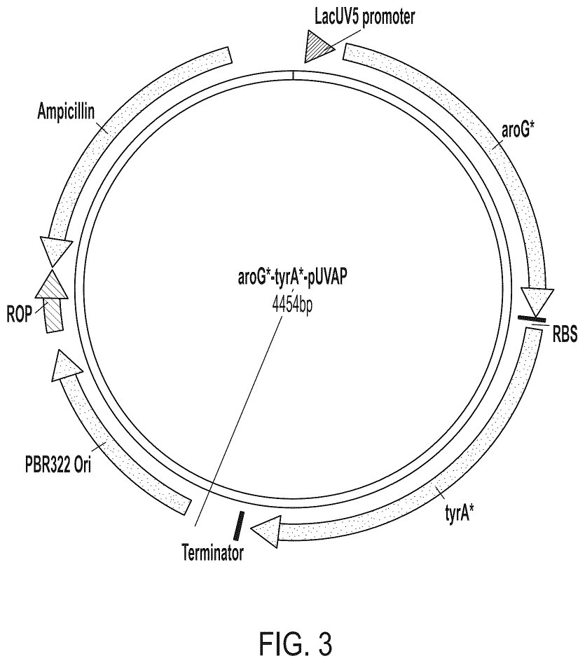

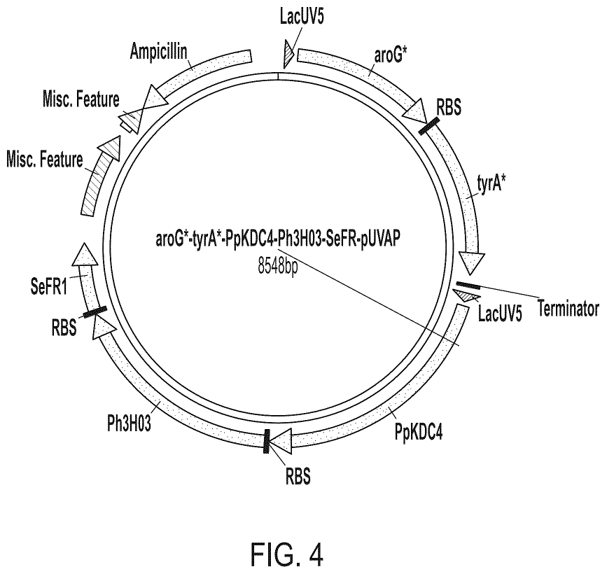

For hydroxytyrosol production, aroG* (SEQ ID NO: 7) and tyrA* (SEQ ID NO: 9) genes were cloned into a pUVAP vector with a Gibson Assembly® cloning kit (New England Biolabs, Massachusetts, U.S.A.), leading to a tyrosine-producing plasmid, aroG*-tyrA*-pUVAP (FIG. 3), in which aroG* and tyrA* are separated by a RBS sequence and the expression of aroG* and tyrA* are controlled by a prokaryotic promoter lacUV5. The plasmid aroG*-tyrA*-pUVAP whole nucleotide sequence is listed in Seq ID No. 11. An operon with PpKDC4 (SEQ ID NO: 3), codon optimized Ph3H03 (SEQ ID NO: 1) and SeFR1 (SEQ ID NO: 5) with a lacUV5 promoter and T7 terminator sequence was then inserted into the aroG*-tyrA*-pUVAP plasmid, generating an aroG*-tyrA*-PpDKC4-Pp3H03-SeFR1-pUVAP plasmid (FIG. 4) for de novo hydroxytyrosol biosynthesis. The complete sequence of the plasmid of FIG. 4 is listed in Seq ID No. 12.

### Example 2: Transformation of the Bacteria

The plasmid of aroG*-tyrA*-PpDKC4-Pp3H03-SeFR1-pUVAP was introduced into E. coli W3110 (DE3) cells by heat shocking a cell and plasmid mixture at 42° C. for 30 seconds, to generate a strain HTYL-01.

### Example 3: Fermentative Production of Hydroxytyrosol

A fermentation process was developed for the de novo production of hydroxytyrosol with the E. coli HTYL strain in fermenters. One mL of HTYL in glycerol stock was inoculated into 100 mL seed culture medium (Luria-Bertani medium with 5 g/L yeast extract, 10 g/L tryptone, 10 g/L NaCl, and 50 mg/L ampicillin) in 500 mL flasks. The seed was cultivated in a shaker at a shaking speed of 200 rpm at 37° C. for 8 hours, and then transferred into 2 liters of fermentation medium A (5 g/L of yeast extract, 0.4 g/L of MgSO₄ 7H₂O, 7.26 g/L of KH₂PO₄, 4 g/L of Na₂HPO₄ 12H₂O, 0.1 g/L of CaCl₂ plus 25 g/L of glucose, 1 ml/L of trace element solution, 2 ml/L of 1% VB1 and 50 mg/L ampicillin) in a 5-liter fermenter.

The HTYL fermenting process in the 5-liter vessel included two phases: a first cell growth phase followed by a de novo biosynthesis phase. The cell growth phase ran from elapsed fermentation time (EFT) 0 hour to about EFT 5 hours. The air flow was set at 0.6 vvm and the pH was not allowed to drop below 7.1 and controlled by additions of aqueous NaOH 4M. The growth temperature was set to 37° C. and the agitation was set to 300-500 rpm. The dissolved oxygen (DO) was cascaded to agitation and maintained above 30%. At EFT 5 hr, isopropyl β-D-1-thiogalactopyranoside (IPTG) was added to a final concentration of 0.5 mM, glucose was fed at a rate of 0.4 g/L/hour, the temperature was decreased to 30° C., and the de novo biosynthesis of hydroxytyrosol commenced on the addition of IPTG.

### Example 4: Product Analysis

During the fermentation process, a portion of fermentation broth was taken and vigorously mixed with 20-50 fold methanol and the mixture was then centrifuged at 20000 g for 15 min. The resultant supernatant was injected into HPLC for analysis.

The HPLC analysis of hydroxytyrosol was carried out on Vanquish Ultimate 3000 system. Intermediates were separated by reverse-phase chromatography on a ACE Excel 2 C18-PFP column (150×2.1 mm id) with a gradient of 0.1% (volume/volume) trifluoric acid (eluant A) and acetonitrile with 0.1% (volume/volume) trifluoric acid (eluant B) in a range of 2 to 45% (vol/vol) eluant B and at a flow rate of 0.25 mil/min. For quantification, all intermediates were calibrated with external standards. The compounds were identified by their retention times, as well as the corresponding spectra, which were identified with a diode array detector in the system.

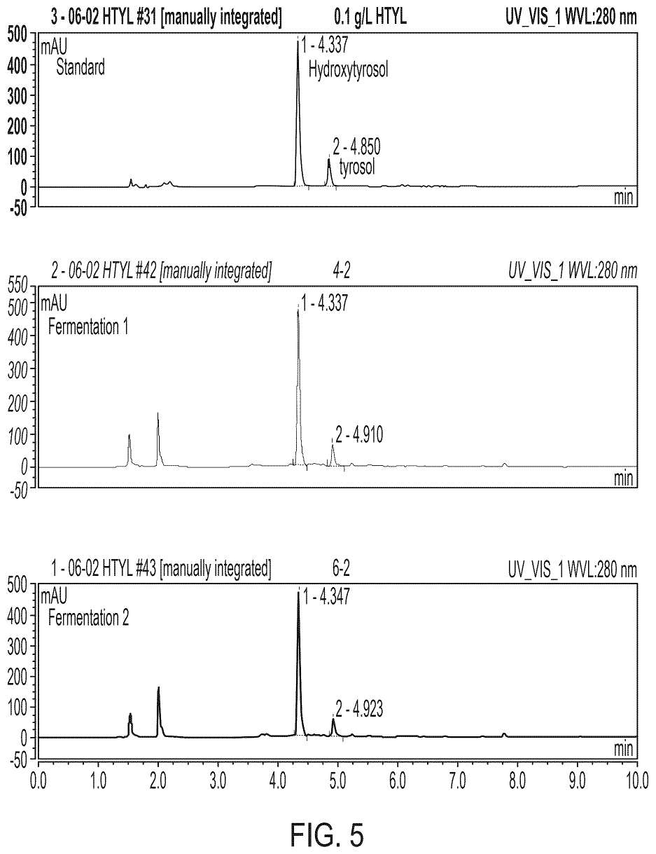

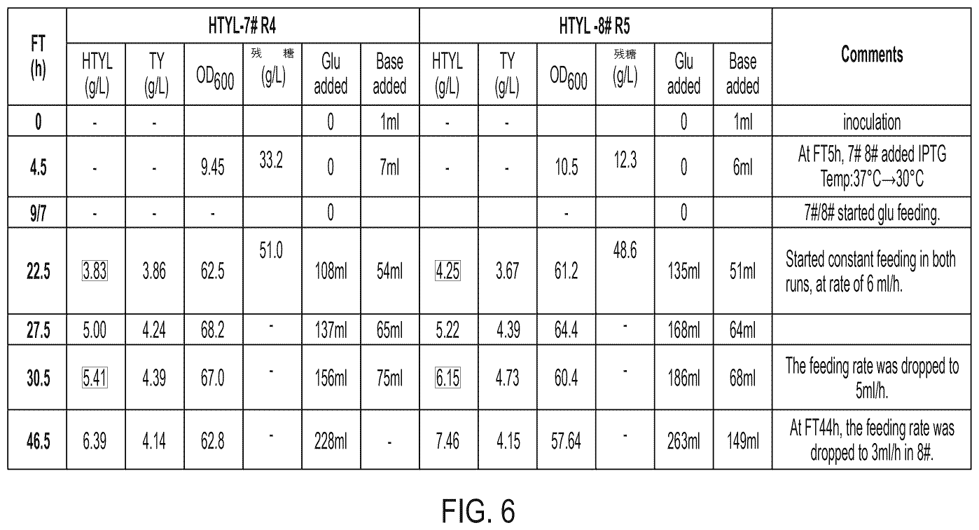

A typical HPLC analytic profile of the fermentation mixture is shown in FIG. 5, and the yields of typical hydroxytyrosol production runs are listed in the table of FIG. 6.

SEQUENCES

SEQ ID NO: 1 DNA sequence encoding (Ph3H03), codon optimized
for E. coli expression
ATGACCACCGCACCGGTTGCCGATAAAATTGCCGAAACCCCGACCGTTACCGCA
GCAAATAATGTGCGCCCGATGACCGGCGCAGAATATCTGGAAAGTCTGCGCGAT
GGTCGTGAAATTTATATTCGTGGTGAACGTGTGGAAGATGTTACCGAACATCCGG
CCTTTCGTAATAGTGCACGTAGTGTGGCCCGTATGTATGATGCACTGCATGAACC
GGCAGCACAGGGTGTGCTGAGCGTTCCGACCGATACCGGTAATGGCGGTTTTAC
CCATCCGTTTTTTAAAACCGCACATAATAGCGATGATCTGATGGCCGCACGTGAT
GCAATTGTTGCCTGGCAGCGTGAAGTTTATGGTTGGCTGGGCCGTAGCCCGGATT
ATAAAGCAAGTTTTCTGGGCACCCTGGGCGCAAATAGCGATTTTTATGGCGAATA
TAAACAGAATGCACTGGATTGGTATAAAAAAAGTCAGGAAAGTGTTCTGTACCT
GAATCATGCCATTGTGAATCCGCCGATTGATCGTGCCAAACCGGCAGATGAAAC
CGCAGATGTGTGCGTTCATGTTGTGGAAGAAACCGATGCAGGCTTAATTGTGAGT
GGTGCAAAAGTGGTTGCCACCGGTAGTGCAATTACCAATGCAAATTTTATTGCCC
ATTACGGTCTGCCGCTGCGCAAAAAAGAATTTGGTCTGATTTTTACCGTGCCGAT
GGATAGTCCGGGCCTGAAACTGCTGTGCCGCACCAGTTATGAAATGAATGCCGC
AGCAACCGGTACACCGTTTGATTATCCGCTGAGCAGTCGTTTTGATGAAAATGAT
AGCATTATGGTTTTCGATCGCGTTCTGGTGCCGTGGGAAAATGTTTTTGCATACG
ATGCAGAAACCACCAATAATTTTGTGATGCGCAGCGGCTTTCTGAATCGCTTTAT
GTTTCATGGCTGCGCCCGCCTGGCAGTTAAACTGGATTTTATTGCCGGCTGTGTG
ATGAAAGGCGTGGAAATGACCGGTACAGCCGGTTTTCGCGGCGTGCAGATGCAG
ATTGGCGAAATTCTGAATTGGCGCGATATGTTTTGGGGTCTGAGTGATGCAATGG
CTAAAAGCCCGGATGAATGGGTGAATGGCGCCGTTCAGCCGAATCTGAATTATG
GTCTGGCATATCGCACCTTTATGGGTGTGGGCTATCCGCGCATTAAAGAAATTAT
TCAGCAGGTGCTGGGCAGCGGCCTGATTTATCTGAATAGTCATGCAGATGATTGG
AAAAATCCTGATATTGAACCGTATCTGAATCAGTATGTGCGTGGTAGCGATGGTA
TTGCCGCAATTGATCGCGTTCAGCTGCTGAAACTGTTATGGGATGCCGTTGGTAC
AGAATTTGGTGGCCGCCATGAACTGTATGAACGTAATTATGGTGGTGATCATGAA
GCCGTTCGCTTTCAGACCCTGTTTGCATATCAGGCCAGCGGTCAGGATGCCGCCC
TGAAAGGCTTTGCAGAACAGTGTATGAGCGAATATGATGTTGATGGTTGGACCC
GTCCTGATCTGATTAATAATGATGATCTGGAAATCGTGTGGAATCGTAAATAA
SEQ ID NO: 2 Amino acid sequence of (Ph3H03)
MTTAPVADKIAETPTVTAANNVRPMTGAEYLESLRDGREIYIRGERVEDVTEHPAFR
NSARSVARMYDALHEPAAQGVLSVPTDTGNGGFTHPFFKTAHNSDDLMAARDAIV
AWQREVYGWLGRSPDYKASFLGTLGANSDFYGEYKQNALDWYKKSQESVLYLNH
AIVNPPIDRAKPADETADVCVHVVEETDAGLIVSGAKVVATGSAITNANFIAHYGLPL
RKKEFGLIFTVPMDSPGLKLLCRTSYEMNAAATGTPFDYPLSSREDENDSIMVEDRVL
VPWENVFAYDAETTNNFVMRSGFLNRFMFHGCARLAVKLDFIAGCVMKGVEMTGT
AGFRGVQMQIGEILNWRDMFWGLSDAMAKSPDEWVNGAVQPNLNYGLAYRTFMG
VGYPRIKEIIQQVLGSGLIYLNSHADDWKNPDIEPYLNQYVRGSDGIAAIDRVQLLKL
LWDAVGTEFGGRHELYERNYGGDHEAVRFQTLFAYQASGQDAALKGFAEQCMSEY
DVDGWTRPDLINNDDLEIVWNRK
SEQ ID NO: 3 DNA sequence encoding (PpKDC4)
ATGGCCCCAGTTAAACAAGACTTTAACATAGACGTTCAAACAATCGAGAACACT
GACATCTCTTTGTCTGAATACATATATCTTAGGATAGCCCAATTGGGTGTCAAGT
CGATCTTTGGTGTCCCAGGTGACTTCAACCTGAATCTTGTGGATGAACTAGACAA
AGTTCCCCAATTGAAATGGATAGGATGTTGTAATGAGCTAAATGCCACTTATGCT
GCTGACGGCTATGCAAAAGCATCAGGAACGATAGGCGTTGTGGTCACTACTTAT
GGTGTGGGTGAGCTAAGCGCCATCAACGGTATAGCAGGAGCATTCGCCGAATAT
GCTCCTGTTCTTCACATTGTAGGCACCTCCGCTATGGCAACAAAACGACTTGAAC
ATGTGCACAACATTCATCATCTTGCAGGGTCTAAGAATTTCTTGGATAGACCAGA
CCATTATATATACGAAAAAATGGTTGATGACATCTGCATTGTCAAAGAGTCTTTA
AGCGAGATCGAAAATGCCTGTGGCCAGATAGATAATGCCATCGTACAGACTTAT
CTGCTTTCCAGACCAGGATACCTATTTTTACCTAGAAATATGGCCACTATGAAAG
TTCCAAGAGAAAGGCTATTCAACCAACCATTAGCCTTGGAAAGAGTTGATCTTCA
CCCCGGTGAAACGCTACAAGTTGTTGAGAAGATCTTGGAAAAGTTCTATCATGCA
AAAGAACCGGCCTTGATTGTGGATTATTTAACCAGACCCTTTAGAATGATGGAAA
ACTGCTCCAAGTTGATCGGTGCTTTGGAGAATAAAGTCAACATTTTCAGCAGACC
AATGTCCAAAGGCTTTGTCGATGAGAGCCACCCAAGATATATTGGCTGTTATATT
GGAAAACAATCCAAGCACCCAAGTACTAGTGATATTCTAGAAAAAAAGAGCGAT
TTCATTCTGAGTGTGGGAACGTTTGATGTTGAAACGAATAACGGAGGCTTCACAT
CCAAGCTTCCCCAAGAACATTTGGTGGAATTGAACCCTCATTTTACTCGTGTTGG
AACTCAATGCTTTTCAAATGTTAACATGTGCCATGTCCTCCCACTCCTGGCAAGT
AAGCTGAGAGGTGATTTGATCAGCATGGCCACAGTTCATCCAAATGATTTCAGCC
TCAGAAAGAAAGAAAAGGCACAGGATAAAATGAAGGCTCTCAACCAGAGCCAT
CTTGTAAAATCTACAGAGCTACTCCTAAATGCAAACGACACTTTGATAGTTGAAA
CTTGCTCATTTATGTTTGCAGTTCCAGATATCGCGTTCCCTAACAATACACAATTT
ATCAGTCAGTCATTCTACAACTCCATAGGATACGCACTTCCCGCCACCTTGGGTG
TCAGCATTGCAAAGCGTGATTTCAGAAAACCAGGAAAAGTCGTACTTATTCAAG
GGGATGGCTCTGCTCAAATGACCATTCAAGAACTGGCTACCATGGTCAGACAAA
AGGTCAAACCCACCATTTTACTCCTCAACAACGAAGGATACACAGTTGAACGAA
TGATTCTAGGTCCAACCAAAGAATATAACGATATAGCTCCTAATTGGGATTGGAC
TGGAATGCTGAGAGCATTCGGCGATATACGTGGCCATTCCAAGAGCATCTCAATT
GACACATGTGGTAGATTAGATAAGCTCGTTCAAACTCGAGAGTTTCAAGAACCT
ACACACCTAAATTTTGTGGAACTTATCTTGGGCAGAATGGACGCCCCGGAAAGG
TTTGCCAATATGGTAAAAGAAATTGCTAACTTAGAGCACGCAAGTAAAAGTATTC
ATTAG
SEQ ID NO: 4 Amino acid sequence of (PpKDC4)
MAPVKQDFNIDVQTIENTDISLSEYTYLRIAQLGVKSIFGVPGDENLNLVDELDKVPQL
KWIGCCNELNATYAADGYAKASGTIGVVVTTYGVGELSAINGIAGAFAEYAPVLHIV
GTSAMATKRLEHVHNIHHLAGSKNFLDRPDHYIYEKMVDDICIVKESLSEIENACGQI
DNAIVQTYLLSRPGYLFLPRNMATMKVPRERLFNQPLALERVDLHPGETLQVVEKIL
EKFYHAKEPALIVDYLTRPFRMMENCSKLIGALENKVNIFSRPMSKGFVDESHPRYIG
CYIGKQSKHPSTSDILEKKSDFILSVGTFDVETNNGGFTSKLPQEHLVELNPHFTRVGT
QCFSNVNMCHVLPLLASKLRGDLISMATVHPNDFSLRKKEKAQDKMKALNQSHLV
KSTELLLNANDTLIVETCSFMFAVPDIAFPNNTQFISQSFYNSIGYALPATLGVSIAKR
DFRKPGKVVLIQGDGSAQMTIQELATMVRQKVKPTILLLNNEGYTVERMILGPTKEY
NDIAPNWDWTGMLRAFGDIRGHSKSISIDTCGRLDKLVQTREFQEPTHLNFVELILGR
MDAPERFANMVKEIANLEHASKSIH
SEQ ID NO: 5 DNA sequence encoding (SeFR1)
ATGATGACCGTTTATGATAGCGCACTGACAATGGAAGAAACCACCCTGCGTGAT
GCAATGAGCCGTTTTGCAACCGGTGTTAGCGTTGTTACCGTTGGTGGTGAACATA
CACATGGTATGACCGCAAATGCCTTTACCTGTGTTAGCCTGGATCCGCCTCTGGT
TCTGTGTTGTGTTGCACGTAAAGCAACCATGCATGCAGCAATTGAAGGTGCACGT
CGTTTTGCAGTTAGCGTTATGGGTGGTGATCAAGAACGTACCGCACGTTATTTTG
CAGATAAACGTCGTCCGCGTGGTCGTGCACAGTTTGATGTTGTTGATTGGCAGCC
TGGTCCGCATACAGGTGCACCGCTGCTGAGCGGTGCGCTGGCATGGCTGGAATG
TGAAGTTGCACAGTGGCATGAAGGTGGCGATCATACCATTTTTCTGGGTCGTGTT
CTGGGTTGTCGTCGTGGTCCGGATAGTCCGGCACTGCTGTTTTATGGTAGCGATTT
TCATCAGATCCGCTAA
SEQ ID NO: 6 Amino acid sequence of (SeFR1)
MMTVYDSALTMEETTLRDAMSRFATGVSVVTVGGEHTHGMTANAFTCVSLDPPLV
LCCVARKATMHAAIEGARRFAVSVMGGDQERTARYFADKRRPRGRAQFDVVDWQ
PGPHTGAPLLSGALAWLECEVAQWHEGGDHTIFLGRVLGCRRGPDSPALLFYGSDF
HQIR
SEQ ID NO: 7 DNA sequence encoding (AroG*)
ATGAATTATCAGAACGACGATTTACGCATCAAAGAAATCAAAGAGTTACTTCCTC
CTGTCGCATTGCTGGAAAAATTCCCCGCTACTGAAAATGCCGCGAATACGGTTGC
CCATGCCCGAAAAGCGATCCATAAGATCCTGAAAGGTAATGATGATCGCCTGTT
GGTTGTGATTGGCCCATGCTCAATTCATGATCCTGTCGCGGCAAAAGAGTATGCC
ACTCGCTTGCTGGCGCTGCGTGAAGAGCTGAAAGATGAGCTGGAAATCGTAATG
CGCGTCTATTTTGAAAAGCCGCGTACCACGGTGGGCTGGAAAGGGCTGATTAAC
GATCCGCATATGGATAATAGCTTCCAGATCAACGACGGTCTGCGTATAGCCCGTA
AATTGCTGCTTGATATTAACGACAGCGGTCTGCCAGCGGCAGGTGAGTTTCTCAA
TATGATCACCCCACAATATCTCGCTGACCTGATGAGCTGGGGCGCAATTGGCGCA
CGTACCACCGAATCGCAGGTGCACCGCGAACTGGCATCAGGGCTTTCTTGTCCGG
TCGGCTTCAAAAATGGCACCGACGGTACGATTAAAGTGGCTATCGATGCCATTA
ATGCCGCCGGTGCGCCGCACTGCTTCCTGTCCGTAACGAAATGGGGGCATTCGGC
GATTGTGAATACCAGCGGTAACGGCGATTGCCATATCATTCTGCGCGGCGGTAA
AGAGCCTAACTACAGCGCGAAGCACGTTGCTGAAGTGAAAGAAGGGCTGAACA
AAGCAGGCCTGCCAGCACAGGTGATGATCGATTTCAGCCATGCTAACTCGTCCA
AACAATTCAAAAAGCAGATGGATGTTTGTGCTGACGTTTGCCAGCAGATTGCCG
GTGGCGAAAAGGCCATTATTGGCGTGATGGTGGAAAGCCATCTGGTGGAAGGCA
ATCAGAGCCTCGAGAGCGGGGAGCCGCTGGCCTACGGTAAGAGCATCACCGATG
CCTGCATCGGCTGGGAAGATACCGATGCTCTGTTACGTCAACTGGCGAATGCAGT
AAAAGCGCGTCGCGGGTAA
SEQ ID NO: 8 Amino acid sequence of (AroG*)
MNYQNDDLRIKEIKELLPPVALLEKFPATENAANTVAHARKAIHKILKGNDDRLLVV
IGPCSIHDPVAAKEYATRLLALREELKDELEIVMRVYFEKPRTTVGWKGLINDPHMD
NSFQINDGLRIARKLLLDINDSGLPAAGEFLNMITPQYLADLMSWGAIGARTTESQVH
RELASGLSCPVGFKNGTDGTIKVAIDAINAAGAPHCFLSVTKWGHSAIVNTSGNGDC
HIILRGGKEPNYSAKHVAEVKEGLNKAGLPAQVMIDFSHANSSKQFKKQMDVCADV
CQQIAGGEKAIIGVMVESHLVEGNQSLESGEPLAYGKSITDACIGWEDTDALLRQLA
NAVKARRG
SEQ ID NO: 9 DNA sequence encoding (TyrA*)
ATGGTTGCTGAATTGACCGCATTACGCGATCAAATTGATGAAGTCGATAAAGCG
CTGCTGAATTTATTAGCGAAGCGTCTGGAACTGGTTGCTGAAGTGGGCGAGGTGA
AAAGCCGCTTTGGACTGCCTATTTATGTTCCGGAGCGCGAGGCATCTATTTTGGC
CTCGCGTCGTGCAGAGGCGGAAGCTCTGGGTGTACCGCCAGATCTGATTGAGGA
TGTTTTGCGTCGGGTGATGCGTGAATCTTACTCCAGTGAAAACGACAAAGGATTT
AAAACACTTTGTCCGTCACTGCGTCCGGTGGTTATCGTCGGCGGTGGCGGTCAGA
TGGGACGCCTGTTCGAGAAGATGCTGACCCTCTCGGGTTATCAGGTGCGGATTCT
GGAGCAACATGACTGGGATCGAGCGGCTGATATTGTTGCCGATGCCGGAATGGT
GATTGTTAGTGTGCCAATCCACGTTACTGAGCAAGTTATTGGCAAATTACCGCCT
TTACCGAAAGATTGTATTCTGGTCGATCTGGCATCAGTGAAAAATGGGCCATTAC
AGGCCATGCTGGTGGCGCATGATGGTCCGGTGCTGGGGCTACACCCGATGTTCG
GTCCGGACAGCGGTAGCCTGGCAAAGCAAGTTGTGGTCTGGTGTGATGGACGTA
AACCGGAAGCATACCAATGGTTTCTGGAGCAAATTCAGGTCTGGGGCGCTCGGC
TGCATCGTATTAGCGCCGTCGAGCACGATCAGAATATGGCGTTTATTCAGGCACT
GCGCCACTTTGCTACTTTTGCTTACGGGCTGCACCTGGCAGAAGAAAATGTTCAG
CTTGAGCAACTTCTGGCGCTCTCTTCGCCGATTTACCGCCTTGAGCTGGCGATGG
TCGGGCGACTGTTTGCTCAGGATCCGCAGCTTTATGCCGACATCATTATGTCGTC
AGAGCGTAATCTGGCGTTAATCAAACGTTACTATAAGCGTTTCGGCGAGGCGATT
GAGTTGCTGGAGCAGGGCGATAAGCAGGCGTTTATTGACAGTTTCCGCAAGGTG
GAGCACTGGTTCGGCGATTACGTACAGCGTTTTCAGAGTGAAAGCCGCGTGTTAT
TGCGTCAGGCGAATGACAATCGCCAGTAA
SEQ ID NO: 10. Amino acid sequence of (TyrA*)
MVAELTALRDQIDEVDKALLNLLAKRLELVAEVGEVKSRFGLPIYVPEREASILASRR
AEAEALGVPPDLIEDVLRRVMRESYSSENDKGFKTLCPSLRPVVIVGGGGQMGRLFE
KMLTLSGYQVRILEQHDWDRAADIVADAGMVIVSVPIHVTEQVIGKLPPLPKDCILV
DLASVKNGPLQAMLVAHDGPVLGLHPMFGPDSGSLAKQVVVWCDGRKPEAYQWF
LEQIQVWGARLHRISAVEHDQNMAFIQALRHFATFAYGLHLAEENVQLEQLLALSSP
IYRLELAMVGRLFAQDPQLYADIIMSSERNLALIKRYYKRFGEAIELLEQGDKQAFID
SFRKVEHWFGDYVQRFQSESRVLLRQANDNRQ
SEQ ID NO: 11 DNA sequence of plasmid vector (aroG*-tyrA*-pUVAP)
GGCAGTGAGCGCAACGCAATTAATGTGAGTTAGCTCACTCATTAGGCACCATCGT
TTAGGCACCCCAGGCTTTACACTTTATGCTTCCGGCTCGTATAATGTGTGGAATT
GTGAGCGGATAACAATTTCAACTATAAGAAGGAGATATACATATGAATTATCAG
AACGACGATTTACGCATCAAAGAAATCAAAGAGTTACTTCCTCCTGTCGCATTGC
TGGAAAAATTCCCCGCTACTGAAAATGCCGCGAATACGGTTGCCCATGCCCGAA
AAGCGATCCATAAGATCCTGAAAGGTAATGATGATCGCCTGTTGGTTGTGATTGG
CCCATGCTCAATTCATGATCCTGTCGCGGCAAAAGAGTATGCCACTCGCTTGCTG
GCGCTGCGTGAAGAGCTGAAAGATGAGCTGGAAATCGTAATGCGCGTCTATTTT
GAAAAGCCGCGTACCACGGTGGGCTGGAAAGGGCTGATTAACGATCCGCATATG
GATAATAGCTTCCAGATCAACGACGGTCTGCGTATAGCCCGTAAATTGCTGCTTG
ATATTAACGACAGCGGTCTGCCAGCGGCAGGTGAGTTTCTCAATATGATCACCCC
ACAATATCTCGCTGACCTGATGAGCTGGGGCGCAATTGGCGCACGTACCACCGA
ATCGCAGGTGCACCGCGAACTGGCATCAGGGCTTTCTTGTCCGGTCGGCTTCAAA
AATGGCACCGACGGTACGATTAAAGTGGCTATCGATGCCATTAATGCCGCCGGT
GCGCCGCACTGCTTCCTGTCCGTAACGAAATGGGGGCATTCGGCGATTGTGAATA
CCAGCGGTAACGGCGATTGCCATATCATTCTGCGCGGCGGTAAAGAGCCTAACT
ACAGCGCGAAGCACGTTGCTGAAGTGAAAGAAGGGCTGAACAAAGCAGGCCTG
CCAGCACAGGTGATGATCGATTTCAGCCATGCTAACTCGTCCAAACAATTCAAAA
AGCAGATGGATGTTTGTGCTGACGTTTGCCAGCAGATTGCCGGTGGCGAAAAGG
CCATTATTGGCGTGATGGTGGAAAGCCATCTGGTGGAAGGCAATCAGAGCCTCG
AGAGCGGGGAGCCGCTGGCCTACGGTAAGAGCATCACCGATGCCTGCATCGGCT
GGGAAGATACCGATGCTCTGTTACGTCAACTGGCGAATGCAGTAAAAGCGCGTC
GCGGGTAACGCAGCAGGAGGTTAAGATGGTTGCTGAATTGACCGCATTACGCGA
TCAAATTGATGAAGTCGATAAAGCGCTGCTGAATTTATTAGCGAAGCGTCTGGAA
CTGGTTGCTGAAGTGGGCGAGGTGAAAAGCCGCTTTGGACTGCCTATTTATGTTC
CGGAGCGCGAGGCATCTATTTTGGCCTCGCGTCGTGCAGAGGCGGAAGCTCTGG
GTGTACCGCCAGATCTGATTGAGGATGTTTTGCGTCGGGTGATGCGTGAATCTTA
CTCCAGTGAAAACGACAAAGGATTTAAAACACTTTGTCCGTCACTGCGTCCGGTG
GTTATCGTCGGCGGTGGCGGTCAGATGGGACGCCTGTTCGAGAAGATGCTGACC
CTCTCGGGTTATCAGGTGCGGATTCTGGAGCAACATGACTGGGATCGAGCGGCT
GATATTGTTGCCGATGCCGGAATGGTGATTGTTAGTGTGCCAATCCACGTTACTG
AGCAAGTTATTGGCAAATTACCGCCTTTACCGAAAGATTGTATTCTGGTCGATCT
GGCATCAGTGAAAAATGGGCCATTACAGGCCATGCTGGTGGCGCATGATGGTCC
GGTGCTGGGGCTACACCCGATGTTCGGTCCGGACAGCGGTAGCCTGGCAAAGCA
AGTTGTGGTCTGGTGTGATGGACGTAAACCGGAAGCATACCAATGGTTTCTGGAG
CAAATTCAGGTCTGGGGCGCTCGGCTGCATCGTATTAGCGCCGTCGAGCACGATC
AGAATATGGCGTTTATTCAGGCACTGCGCCACTTTGCTACTTTTGCTTACGGGCT
GCACCTGGCAGAAGAAAATGTTCAGCTTGAGCAACTTCTGGCGCTCTCTTCGCCG
ATTTACCGCCTTGAGCTGGCGATGGTCGGGCGACTGTTTGCTCAGGATCCGCAGC
TTTATGCCGACATCATTATGTCGTCAGAGCGTAATCTGGCGTTAATCAAACGTTA
CTATAAGCGTTTCGGCGAGGCGATTGAGTTGCTGGAGCAGGGCGATAAGCAGGC
GTTTATTGACAGTTTCCGCAAGGTGGAGCACTGGTTCGGCGATTACGTACAGCGT
TTTCAGAGTGAAAGCCGCGTGTTATTGCGTCAGGCGAATGACAATCGCCAGTAA
GCGGCCGCCACCGCTGAGCAATAACTAGCATAACCCCTTGGGGCCTCTAAACGG
GTCTTGAGGGGTTTTTTGCTGAAAGGAGGAACTATATCCGGGTAACGAATTCAAG
CTTGATATCATTCAGGACGAGCCTCAGACTCCAGCGTAACTGGACTGCAATCAAC
TCACTGGCTCACCTTCACGGGTGGGCCTTTCTTCGGTAGAAAATCAAAGGATCTT
CTTGAGATCCTTTTTTTCTGCGCGTAATCTGCTGCTTGCAAACAAAAAAACCACC
GCTACCAGCGGTGGTTTGTTTGCCGGATCAAGAGCTACCAACTCTTTTTCCGAGG
TAACTGGCTTCAGCAGAGCGCAGATACCAAATACTGTTCTTCTAGTGTAGCCGTA
GTTAGGCCACCACTTCAAGAACTCTGTAGCACCGCCTACATACCTCGCTCTGCTA
ATCCTGTTACCAGTGGCTGCTGCCAGTGGCGATAAGTCGTGTCTTACCGGGTTGG
ACTCAAGACGATAGTTACCGGATAAGGCGCAGCGGTCGGGCTGAACGGGGGGTT
CGTGCACACAGCCCAGCTTGGAGCGAACGACCTACACCGAACTGAGATACCTAC
AGCGTGAGCTATGAGAAAGCGCCACGCTTCCCGAAGGGAGAAAGGCGGACAGG
TATCCGGTAAGCGGCAGGGTCGGAACAGGAGAGCGCACGAGGGAGCTTCCAGG
GGGAAACGCCTGGTATCTTTATAGTCCTGTCGGGTTTCGCCACCTCTGACTTGAG
CATCGATTTTTGTGATGCTCGTCAGGGGGGCGGAGCCTATGGAAAAACGCCAGC
AACGCAGAAAGGCCCACCCGAAGGTGAGCCAGGTGATTACATTTGGGCCCTCAT
CAGAGGTTTTCACCGTCATCACCGAAACGCGCGAGGCAGCTGCGGTAAAGCTCA
TCAGCGTGGTCGTGAAGCGATTCACAGATGTCTGCCTGTTCATCCGCGTCCAGCT
CGTTGAGTTTCTCCAGAAGCGTTAATGTCTGGCTTCTGATAAAGCGGGCCATGTT
AAGGGCGGTTTTTTCCTGTTTGGTCATTTACCAATGCTTAATCAGTGAGGCACCTA
TCTCAGCGATCTGTCTATTTCGTTCATCCATAGTTGCCTGACTCCCCGTCGTGTAG
ATAACTACGATACGGGAGGGCTTACCATCTGGCCCCAGTGCTGCAATGATACCG
CGAGACCCACGCTCACCGGCTCCAGATTTATCAGCAATAAACCAGCCAGCCGGA
AGGGCCGAGCGCAGAAGTGGTCCTGCAACTTTATCCGCCTCCATCCAGTCTATTA
ATTGTTGCCGGGAAGCTAGAGTAAGTAGTTCGCCAGTTAATAGTTTGCGCAACGT
TGTTGCCATTGCTACAGGCATCGTGGTGTCACGCTCGTCGTTTGGTATGGCTTCAT
TCAGCTCCGGTTCCCAACGATCAAGGCGAGTTACATGATCCCCCATGTTGTGCAA
AAAAGCGGTTAGCTCCTTCGGTCCTCCGATCGTTGTCAGAAGTAAGTTGGCCGCA
GTGTTATCACTCATGGTTATGGCAGCACTGCATAATTCTCTTACTGTCATGCCATC
CGTAAGATGCTTTTCTGTGACTGGTGAGTACTCAACCAAGTCATTCTGAGAATAG
TGTATGCGGCGACCGAGTTGCTCTTGCCCGGCGTCAATACGGGATAATACCGCGC
CACATAGCAGAACTTTAAAAGTGCTCATCATTGGAAAACGTTCTTCGGGGCGAA
AACTCTCAAGGATCTTACCGCTGTTGAGATCCAGTTCGATGTAACCCACTCGTGC
ACCCAACTGATCTTCAGCATCTTTTACTTTCACCAGCGTTTCTGGGTGAGCAAAA
ACAGGAAGGCAAAATGCCGCAAAAAAGGGAATAAGGGCGACACGGAAATGTTG
AATACTCATAGCTCCTGAAAATCTCGATAACTCAAAAAATACGCCCGGTAGTGAT
CTTATTTCATTATGGTGAAAGTTGGAACCTCTTACGTGCCGATCAAGTCAAAAGC
CTCCGGTCGGAGGCTTTTGACTTTCTGCTATGGAGGTCAGGTATGATTTAAATGG
TCAGTATTGAGCGATATCTAGAGAATTCGTC
SEQ ID NO: 12 DNA sequence of plasmid vector
(aroG*-tyrA*-PpKDC4-Ph3H03-SeFR1-pUVAP)
GGCAGTGAGCGCAACGCAATTAATGTGAGTTAGCTCACTCATTAGGCACCATCGT
TTAGGCACCCCAGGCTTTACACTTTATGCTTCCGGCTCGTATAATGTGTGGAATT
GTGAGCGGATAACAATTTCAACTATAAGAAGGAGATATACATATGAATTATCAG
AACGACGATTTACGCATCAAAGAAATCAAAGAGTTACTTCCTCCTGTCGCATTGC
TGGAAAAATTCCCCGCTACTGAAAATGCCGCGAATACGGTTGCCCATGCCCGAA
AAGCGATCCATAAGATCCTGAAAGGTAATGATGATCGCCTGTTGGTTGTGATTGG
CCCATGCTCAATTCATGATCCTGTCGCGGCAAAAGAGTATGCCACTCGCTTGCTG
GCGCTGCGTGAAGAGCTGAAAGATGAGCTGGAAATCGTAATGCGCGTCTATTTT
GAAAAGCCGCGTACCACGGTGGGCTGGAAAGGGCTGATTAACGATCCGCATATG
GATAATAGCTTCCAGATCAACGACGGTCTGCGTATAGCCCGTAAATTGCTGCTTG
ATATTAACGACAGCGGTCTGCCAGCGGCAGGTGAGTTTCTCAATATGATCACCCC
ACAATATCTCGCTGACCTGATGAGCTGGGGCGCAATTGGCGCACGTACCACCGA
ATCGCAGGTGCACCGCGAACTGGCATCAGGGCTTTCTTGTCCGGTCGGCTTCAAA
AATGGCACCGACGGTACGATTAAAGTGGCTATCGATGCCATTAATGCCGCCGGT
GCGCCGCACTGCTTCCTGTCCGTAACGAAATGGGGGCATTCGGCGATTGTGAATA
CCAGCGGTAACGGCGATTGCCATATCATTCTGCGCGGCGGTAAAGAGCCTAACT
ACAGCGCGAAGCACGTTGCTGAAGTGAAAGAAGGGCTGAACAAAGCAGGCCTG
CCAGCACAGGTGATGATCGATTTCAGCCATGCTAACTCGTCCAAACAATTCAAAA
AGCAGATGGATGTTTGTGCTGACGTTTGCCAGCAGATTGCCGGTGGCGAAAAGG
CCATTATTGGCGTGATGGTGGAAAGCCATCTGGTGGAAGGCAATCAGAGCCTCG
AGAGCGGGGAGCCGCTGGCCTACGGTAAGAGCATCACCGATGCCTGCATCGGCT
GGGAAGATACCGATGCTCTGTTACGTCAACTGGCGAATGCAGTAAAAGCGCGTC
GCGGGTAACGCAGCAGGAGGTTAAGATGGTTGCTGAATTGACCGCATTACGCGA
TCAAATTGATGAAGTCGATAAAGCGCTGCTGAATTTATTAGCGAAGCGTCTGGAA
CTGGTTGCTGAAGTGGGCGAGGTGAAAAGCCGCTTTGGACTGCCTATTTATGTTC
CGGAGCGCGAGGCATCTATTTTGGCCTCGCGTCGTGCAGAGGCGGAAGCTCTGG
GTGTACCGCCAGATCTGATTGAGGATGTTTTGCGTCGGGTGATGCGTGAATCTTA
CTCCAGTGAAAACGACAAAGGATTTAAAACACTTTGTCCGTCACTGCGTCCGGTG
GTTATCGTCGGCGGTGGCGGTCAGATGGGACGCCTGTTCGAGAAGATGCTGACC
CTCTCGGGTTATCAGGTGCGGATTCTGGAGCAACATGACTGGGATCGAGCGGCT
GATATTGTTGCCGATGCCGGAATGGTGATTGTTAGTGTGCCAATCCACGTTACTG
AGCAAGTTATTGGCAAATTACCGCCTTTACCGAAAGATTGTATTCTGGTCGATCT
GGCATCAGTGAAAAATGGGCCATTACAGGCCATGCTGGTGGCGCATGATGGTCC
GGTGCTGGGGCTACACCCGATGTTCGGTCCGGACAGCGGTAGCCTGGCAAAGCA
AGTTGTGGTCTGGTGTGATGGACGTAAACCGGAAGCATACCAATGGTTTCTGGAG
CAAATTCAGGTCTGGGGCGCTCGGCTGCATCGTATTAGCGCCGTCGAGCACGATC
AGAATATGGCGTTTATTCAGGCACTGCGCCACTTTGCTACTTTTGCTTACGGGCT
GCACCTGGCAGAAGAAAATGTTCAGCTTGAGCAACTTCTGGCGCTCTCTTCGCCG
ATTTACCGCCTTGAGCTGGCGATGGTCGGGCGACTGTTTGCTCAGGATCCGCAGC
TTTATGCCGACATCATTATGTCGTCAGAGCGTAATCTGGCGTTAATCAAACGTTA
CTATAAGCGTTTCGGCGAGGCGATTGAGTTGCTGGAGCAGGGCGATAAGCAGGC
GTTTATTGACAGTTTCCGCAAGGTGGAGCACTGGTTCGGCGATTACGTACAGCGT
TTTCAGAGTGAAAGCCGCGTGTTATTGCGTCAGGCGAATGACAATCGCCAGTAA
GCGGCCGCCACCGCTGAGCAATAACTAGCATAACCCCTTGGGGCCTCTAAACGG
GTCTTGAGGGGTTTTTTGCTGAAAGGAGGAACTATATCCATCGTTTAGGCACCCC
AGGCTTTACACTTTATGCTTCCGGCTCGTATAATGTGTGGAATTGTGAGCGGATA
ACAATTTCAACTATAAGAAGGAGATATACATATGGCCCCAGTTAAACAAGACTT
TAACATAGACGTTCAAACAATCGAGAACACTGACATCTCTTTGTCTGAATACATA
TATCTTAGGATAGCCCAATTGGGTGTCAAGTCGATCTTTGGTGTCCCAGGTGACT
TCAACCTGAATCTTGTGGATGAACTAGACAAAGTTCCCCAATTGAAATGGATAG
GATGTTGTAATGAGCTAAATGCCACTTATGCTGCTGACGGCTATGCAAAAGCATC
AGGAACGATAGGCGTTGTGGTCACTACTTATGGTGTGGGTGAGCTAAGCGCCAT
CAACGGTATAGCAGGAGCATTCGCCGAATATGCTCCTGTTCTTCACATTGTAGGC
ACCTCCGCTATGGCAACAAAACGACTTGAACATGTGCACAACATTCATCATCTTG
CAGGGTCTAAGAATTTCTTGGATAGACCAGACCATTATATATACGAAAAAATGGT
TGATGACATCTGCATTGTCAAAGAGTCTTTAAGCGAGATCGAAAATGCCTGTGGC
CAGATAGATAATGCCATCGTACAGACTTATCTGCTTTCCAGACCAGGATACCTAT
TTTTACCTAGAAATATGGCCACTATGAAAGTTCCAAGAGAAAGGCTATTCAACCA
ACCATTAGCCTTGGAAAGAGTTGATCTTCACCCCGGTGAAACGCTACAAGTTGTT
GAGAAGATCTTGGAAAAGTTCTATCATGCAAAAGAACCGGCCTTGATTGTGGATT
ATTTAACCAGACCCTTTAGAATGATGGAAAACTGCTCCAAGTTGATCGGTGCTTT
GGAGAATAAAGTCAACATTTTCAGCAGACCAATGTCCAAAGGCTTTGTCGATGA
GAGCCACCCAAGATATATTGGCTGTTATATTGGAAAACAATCCAAGCACCCAAG
TACTAGTGATATTCTAGAAAAAAAGAGCGATTTCATTCTGAGTGTGGGAACGTTT
GATGTTGAAACGAATAACGGAGGCTTCACATCCAAGCTTCCCCAAGAACATTTG
GTGGAATTGAACCCTCATTTTACTCGTGTTGGAACTCAATGCTTTTCAAATGTTAA
CATGTGCCATGTCCTCCCACTCCTGGCAAGTAAGCTGAGAGGTGATTTGATCAGC
ATGGCCACAGTTCATCCAAATGATTTCAGCCTCAGAAAGAAAGAAAAGGCACAG
GATAAAATGAAGGCTCTCAACCAGAGCCATCTTGTAAAATCTACAGAGCTACTC
CTAAATGCAAACGACACTTTGATAGTTGAAACTTGCTCATTTATGTTTGCAGTTCC
AGATATCGCGTTCCCTAACAATACACAATTTATCAGTCAGTCATTCTACAACTCC
ATAGGATACGCACTTCCCGCCACCTTGGGTGTCAGCATTGCAAAGCGTGATTTCA
GAAAACCAGGAAAAGTCGTACTTATTCAAGGGGATGGCTCTGCTCAAATGACCA
TTCAAGAACTGGCTACCATGGTCAGACAAAAGGTCAAACCCACCATTTTACTCCT
CAACAACGAAGGATACACAGTTGAACGAATGATTCTAGGTCCAACCAAAGAATA
TAACGATATAGCTCCTAATTGGGATTGGACTGGAATGCTGAGAGCATTCGGCGAT
ATACGTGGCCATTCCAAGAGCATCTCAATTGACACATGTGGTAGATTAGATAAGC
TCGTTCAAACTCGAGAGTTTCAAGAACCTACACACCTAAATTTTGTGGAACTTAT
CTTGGGCAGAATGGACGCCCCGGAAAGGTTTGCCAATATGGTAAAAGAAATTGC
TAACTTAGAGCACGCAAGTAAAAGTATTCATTAGCGCAGCAGGAGGTTAAGATG
ACCACCGCACCGGTTGCCGATAAAATTGCCGAAACCCCGACCGTTACCGCAGCA
AATAATGTGCGCCCGATGACCGGCGCAGAATATCTGGAAAGTCTGCGCGATGGT
CGTGAAATTTATATTCGTGGTGAACGTGTGGAAGATGTTACCGAACATCCGGCCT
TTCGTAATAGTGCACGTAGTGTGGCCCGTATGTATGATGCACTGCATGAACCGGC
AGCACAGGGTGTGCTGAGCGTTCCGACCGATACCGGTAATGGCGGTTTTACCCAT
CCGTTTTTTAAAACCGCACATAATAGCGATGATCTGATGGCCGCACGTGATGCAA
TTGTTGCCTGGCAGCGTGAAGTTTATGGTTGGCTGGGCCGTAGCCCGGATTATAA
AGCAAGTTTTCTGGGCACCCTGGGCGCAAATAGCGATTTTTATGGCGAATATAAA
CAGAATGCACTGGATTGGTATAAAAAAAGTCAGGAAAGTGTTCTGTACCTGAAT
CATGCCATTGTGAATCCGCCGATTGATCGTGCCAAACCGGCAGATGAAACCGCA
GATGTGTGCGTTCATGTTGTGGAAGAAACCGATGCAGGCTTAATTGTGAGTGGTG
CAAAAGTGGTTGCCACCGGTAGTGCAATTACCAATGCAAATTTTATTGCCCATTA
CGGTCTGCCGCTGCGCAAAAAAGAATTTGGTCTGATTTTTACCGTGCCGATGGAT
AGTCCGGGCCTGAAACTGCTGTGCCGCACCAGTTATGAAATGAATGCCGCAGCA
ACCGGTACACCGTTTGATTATCCGCTGAGCAGTCGTTTTGATGAAAATGATAGCA
TTATGGTTTTCGATCGCGTTCTGGTGCCGTGGGAAAATGTTTTTGCATACGATGCA
GAAACCACCAATAATTTTGTGATGCGCAGCGGCTTTCTGAATCGCTTTATGTTTC
ATGGCTGCGCCCGCCTGGCAGTTAAACTGGATTTTATTGCCGGCTGTGTGATGAA
AGGCGTGGAAATGACCGGTACAGCCGGTTTTCGCGGCGTGCAGATGCAGATTGG
CGAAATTCTGAATTGGCGCGATATGTTTTGGGGTCTGAGTGATGCAATGGCTAAA
AGCCCGGATGAATGGGTGAATGGCGCCGTTCAGCCGAATCTGAATTATGGTCTG
GCATATCGCACCTTTATGGGTGTGGGCTATCCGCGCATTAAAGAAATTATTCAGC
AGGTGCTGGGCAGCGGCCTGATTTATCTGAATAGTCATGCAGATGATTGGAAAA
ATCCTGATATTGAACCGTATCTGAATCAGTATGTGCGTGGTAGCGATGGTATTGC
CGCAATTGATCGCGTTCAGCTGCTGAAACTGTTATGGGATGCCGTTGGTACAGAA
TTTGGTGGCCGCCATGAACTGTATGAACGTAATTATGGTGGTGATCATGAAGCCG
TTCGCTTTCAGACCCTGTTTGCATATCAGGCCAGCGGTCAGGATGCCGCCCTGAA
AGGCTTTGCAGAACAGTGTATGAGCGAATATGATGTTGATGGTTGGACCCGTCCT
GATCTGATTAATAATGATGATCTGGAAATCGTGTGGAATCGTAAATAACGCAGC
AGGAGGTTAAGATGATGACCGTTTATGATAGCGCACTGACAATGGAAGAAACCA
CCCTGCGTGATGCAATGAGCCGTTTTGCAACCGGTGTTAGCGTTGTTACCGTTGG
TGGTGAACATACACATGGTATGACCGCAAATGCCTTTACCTGTGTTAGCCTGGAT
CCGCCTCTGGTTCTGTGTTGTGTTGCACGTAAAGCAACCATGCATGCAGCAATTG
AAGGTGCACGTCGTTTTGCAGTTAGCGTTATGGGTGGTGATCAAGAACGTACCGC
ACGTTATTTTGCAGATAAACGTCGTCCGCGTGGTCGTGCACAGTTTGATGTTGTT
GATTGGCAGCCTGGTCCGCATACAGGTGCACCGCTGCTGAGCGGTGCGCTGGCA
TGGCTGGAATGTGAAGTTGCACAGTGGCATGAAGGTGGCGATCATACCATTTTTC
TGGGTCGTGTTCTGGGTTGTCGTCGTGGTCCGGATAGTCCGGCACTGCTGTTTTAT
GGTAGCGATTTTCATCAGATCCGCTAAGCGGCCGCCACCGCTGAGCAATAACTA
GCATAACCCCTTGGGGCCTCTAAACGGGGGGTAACGAATTCAAGCTTGATATCAT
TCAGGACGAGCCTCAGACTCCAGCGTAACTGGACTGCAATCAACTCACTGGCTC
ACCTTCACGGGTGGGCCTTTCTTCGGTAGAAAATCAAAGGATCTTCTTGAGATCC
TTTTTTTCTGCGCGTAATCTGCTGCTTGCAAACAAAAAAACCACCGCTACCAGCG
GTGGTTTGTTTGCCGGATCAAGAGCTACCAACTCTTTTTCCGAGGTAACTGGCTT
CAGCAGAGCGCAGATACCAAATACTGTTCTTCTAGTGTAGCCGTAGTTAGGCCAC
CACTTCAAGAACTCTGTAGCACCGCCTACATACCTCGCTCTGCTAATCCTGTTAC
CAGTGGCTGCTGCCAGTGGCGATAAGTCGTGTCTTACCGGGTTGGACTCAAGACG
ATAGTTACCGGATAAGGCGCAGCGGTCGGGCTGAACGGGGGGTTCGTGCACACA
GCCCAGCTTGGAGCGAACGACCTACACCGAACTGAGATACCTACAGCGTGAGCT
ATGAGAAAGCGCCACGCTTCCCGAAGGGAGAAAGGCGGACAGGTATCCGGTAA
GCGGCAGGGTCGGAACAGGAGAGCGCACGAGGGAGCTTCCAGGGGGAAACGCC
TGGTATCTTTATAGTCCTGTCGGGTTTCGCCACCTCTGACTTGAGCATCGATTTTT
GTGATGCTCGTCAGGGGGGCGGAGCCTATGGAAAAACGCCAGCAACGCAGAAA
GGCCCACCCGAAGGTGAGCCAGGTGATTACATTTGGGCCCTCATCAGAGGTTTTC
ACCGTCATCACCGAAACGCGCGAGGCAGCTGCGGTAAAGCTCATCAGCGTGGTC
GTGAAGCGATTCACAGATGTCTGCCTGTTCATCCGCGTCCAGCTCGTTGAGTTTC
TCCAGAAGCQTTAATGTCTGGCTTCTGATAAAGCGGGCCATGTTAAGGGCGGTTT
TTTCCTGTTTGGTCATTTACCAATGCTTAATCAGTGAGGCACCTATCTCAGCGATC
TGTCTATTTCGTTCATCCATAGTTGCCTGACTCCCCGTCGTGTAGATAACTACGAT
ACGGGAGGGCTTACCATCTGGCCCCAGTGCTGCAATGATACCGCGAGACCCACG
CTCACCGGCTCCAGATTTATCAGCAATAAACCAGCCAGCCGGAAGGGCCGAGCG
CAGAAGTGGTCCTGCAACTTTATCCGCCTCCATCCAGTCTATTAATTGTTGCCGG
GAAGCTAGAGTAAGTAGTTCGCCAGTTAATAGTTTGCGCAACGTTGTTGCCATTG
CTACAGGCATCGTGGTGTCACGCTCGTCGTTTGGTATGGCTTCATTCAGCTCCGG
TTCCCAACGATCAAGGCGAGTTACATGATCCCCCATGTTGTGCAAAAAAGCGGTT
AGCTCCTTCGGTCCTCCGATCGTTGTCAGAAGTAAGTTGGCCGCAGTGTTATCAC
TCATGGTTATGGCAGCACTGCATAATTCTCTTACTGTCATGCCATCCGTAAGATG
CTTTTCTGTGACTGGTGAGTACTCAACCAAGTCATTCTGAGAATAGTGTATGCGG
CGACCGAGTTGCTCTTGCCCGGCGTCAATACGGGATAATACCGCGCCACATAGC
AGAACTTTAAAAGTGCTCATCATTGGAAAACGTTCTTCGGGGCGAAAACTCTCAA
GGATCTTACCGCTGTTGAGATCCAGTTCGATGTAACCCACTCGTGCACCCAACTG
ATCTTCAGCATCTTTTACTTTCACCAGCGTTTCTGGGTGAGCAAAAACAGGAAGG
CAAAATGCCGCAAAAAAGGGAATAAGGGCGACACGGAAATGTTGAATACTCAT
AGCTCCTGAAAATCTCGATAACTCAAAAAATACGCCCGGTAGTGATCTTATTTCA
TTATGGTGAAAGTTGGAACCTCTTACGTGCCGATCAAGTCAAAAGCCTCCGGTCG
GAGGCTTTTGACTTTCTGCTATGGAGGTCAGGTATGATTTAAATGGTCAGTATTG
AGCGATATCTAGAGAATTCGTC

1. A method of producing hydroxytyrosol, the method comprising:
(i) culturing a recombinant host microorganism in a medium, wherein:
the host microorganism has been transformed with:
a first polynucleotide sequence encoding a decarboxylase enzyme capable of decarboxylating 4-hydroxyphenylpyruvaic acid (HPP) to produce 4-hydroxyphenylacetaldehyde, and
a second polynucleotide sequence encoding a hydroxylase enzyme, wherein
the hydroxylase has an amino acid sequence having at least 90% identity to the amino acid sequence as set forth in SEQ ID NO: 2, and
the medium comprises glucose; and
(ii) collecting product hydroxytyrosol from one or both of the host microorganism and the culture medium.

2. The method according to claim 1, wherein the hydroxylase has an amino acid sequence having at least 95% identity to the amino acid sequence as set forth in SEQ ID NO: 2.

3. The method of claim 1, wherein the hydroxylase has an amino acid sequence with at least 99% identity to the amino acid sequence as set forth in SEQ ID NO: 2.

4. The method according to claim 1, wherein the decarboxylase has an amino acid sequence with at least 95% identity to the amino acid sequence as set forth in SEQ ID NO: 4.

5. The method according to claim 1, wherein the decarboxylase has an amino acid sequence having at least 99% identity to the amino acid sequence as set forth in SEQ ID NO: 4.

6. The method according to claim 1, wherein the host microorganism has been further transformed to overexpress a DAHP synthase relative to a corresponding parental host cell.

7. The method according to claim 6, wherein the DAHP synthase is AroG*.

8. The method according to claim 1, wherein the host microorganism has been further transformed to overexpress an enzyme relative to a corresponding parental host cell, wherein the catalytic activity of the overexpressed enzyme includes converting chorismate (CHA) to prephenate (PPA) relative to a parental host cell.

9. The method according to claim 8, wherein the enzyme whose catalytic activity includes converting chorismate to prephenate is TyrA*.

10. A recombinant host microorganism comprising a metabolic pathway producing hydroxytyrosol, wherein the host microorganism has been transformed with a first polynucleotide sequence encoding a decarboxylase enzyme capable of decarboxylating 4-hydroxyphenylpyruvaic acid (HPP) to produce 4-hydroxyphenylacetaldehyde, and a second polynucleotide sequence encoding a hydroxylase enzyme, wherein the hydroxylase has an amino acid sequence with at least 90% identity to the amino acid sequence as set forth in SEQ ID NO: 2.

11. The recombinant host microorganism according to claim 10, wherein the hydroxylase has an amino acid sequence having at least 95% identity to the amino acid sequence as set forth in SEQ ID NO: 2.

12. The recombinant host microorganism according to claim 10, wherein the hydroxylase has an amino acid sequence having at least 99% identity to the amino acid sequence as set forth SEQ ID NO: 2.

13. The recombinant host microorganism according to claim 10, wherein the decarboxylase has an amino acid sequence having at least 95% identity to the amino acid sequence as set forth in SEQ ID NO: 4.

14. The recombinant host microorganism according to claim 10, wherein the decarboxylase has an amino acid sequence having at least 99% identity to the amino acid sequence as set forth in SEQ ID NO: 4.

15. The recombinant host microorganism according to claim 10, wherein the microorganism has been further transformed to overexpress a DAHP synthase relative to a corresponding parental host cell.

16. The recombinant host microorganism according to claim 15, wherein the DAHP synthase is AroG*.

17. The recombinant host microorganism according to claim 10, wherein the microorganism has been further transformed to overexpress an enzyme relative to a corresponding parental host cell, wherein the catalytic activity of the overexpressed enzyme includes converting chorismate (CHA) to prephenate (PPA) relative to a parental host cell.

18. The recombinant host microorganism according to claim 17, wherein the enzyme whose catalytic activity includes converting chorismate to prephenate is TyrA*.

19. The recombinant host microorganism according to claim 10, wherein the host microorganism is of a genus selected from the group Escherichia, Bacillus, Corynebacterium, Saccharomyces, and Yarrowia.

20. The recombinant host microorganism according to claim 10, wherein the host microorganism is Escherichia coli.
